(19) United States
(12) Patent Application Publication
(10) Pub. No.: US 2025/0115898 A1
(43) Pub. Date: Apr. 10, 2025

(54) SYSTEMS AND METHODS FOR SIMULTANEOUS DETECTION OF ANTIGENS AND LIGANDS THEREOF

(71) Applicant: Vanderbilt University, Nashville, TN (US)

(72) Inventors: Hema Preethi SUBAS SATISH, Nashville, TN (US); Ivelin GEORGIEV, Nashville, TN (US)

(21) Appl. No.: 18/907,125

(22) Filed: Oct. 4, 2024

Related U.S. Application Data
(60) Provisional application No. 63/588,443, filed on Oct. 6, 2023.

(51) Int. Cl. C12N 15/10; C12Q 1/6806; C12Q 1/686; C12Q 1/6869; G01N 33/68

(52) U.S. Cl. CPC C12N 15/1037; C12N 15/1096; C12Q 1/6806; C12Q 1/686; C12Q 1/6869; G01N 33/6854

(57) ABSTRACT

The present disclosure relates to a system for simultaneous detection of antigens and antigen specific ligands, and methods of use thereof.

### CROSS REFERENCE TO RELATED APPLICATION

This application claims priority to and the benefit of U.S. Provisional Application No. 63/588,443, entitled “SYSTEMS AND METHODS FOR SIMULTANEOUS DETECTION OF ANTIGENS AND LIGANDS THEREOF,” filed on Oct. 6, 2023, the contents of which are hereby incorporated by reference in their entirety.

### STATEMENT REGARDING FEDERALLY SPONSORED RESEARCH

This invention was made with government support under Grant Nos. R01 A1175245 and R01 AI152693 awarded by the National Institutes of Health. The government has certain rights in the invention.

### REFERENCE TO SEQUENCE LISTING

The Sequence Listing conforming to the rules of WIPO Standard ST.26 is hereby incorporated by reference. Said Sequence Listing has been filed as an electronic document via Patent Center encoded as XML in UTF-8 text. The electronic document, created on Oct. 4, 2024, is entitled “10644-172US1 Sequence Listing.xml,” and is 550,663 bytes in size.

### FIELD

The present disclosure relates to systems and methods for simultaneous detection of antigens and antigen specific ligands.

### BACKGROUND

The human immune system participates in complex interactions with virtually all other systems in the body. In particular, the B-cell component of the adaptive immune response plays a role in various disease settings, including infectious disease, cancer, autoimmunity, cardiovascular, hematologic, and neurologic diseases. In addition, antibodies (a product of B cells) are effectively used in diagnostics and therapeutics. Despite decades of antibody discovery efforts, there is still limited public data linking human antibody sequences to antigen specificity. This is because the processes of obtaining antibody sequence and binding information are typically decoupled and low throughput. While next-generation sequencing (NGS) of B-cell receptor (BCR) transcripts enables high-throughput interrogation of the BCR sequence repertoire, uniting these BCR sequence with cognate antigen specificity requires the production and characterization of sequences of interest one at a time.

To date, the process of isolating potent neutralizing antibodies from human samples or immunized animal models has been driven by a variety of methods like single-cell sorting, screens of immortalized B cells or hybridomas, and B cell culture. However, initial screening of cell supernatants is low-throughput and typically restricted to few antigens, which limits the number of antibodies that can be characterized and the ability to detect cross-reactivity. An alternative to screening supernatants of cultured or immortalized B cells is to screen surface-expressed BCRs via antigen-specific B cell sorting. In antigen-specific B cell sorting, an antigen is conjugated to a fluorophore and used to stain a B cell population of interest. This allows the identification of rare clones, but the subsequent determination of BCR sequences requires cloning V_{H} and V_{κ}/V_{λ} genes and Sanger sequencing for each individual B cell. Notably, none of these traditional methods are conducive to recovering antibody sequence and function simultaneously.

### SUMMARY

Disclosed herein are systems and methods for simultaneous detection of antigens and antigen specific antibodies, ligands, or binding fragment thereof.

Various aspects include a method for simultaneous detection of an antigen and an antibody or binding fragment thereof that specifically binds said antigen. In some aspects, the method includes: contacting a population of eukaryotic cells engineered to display a plurality of barcode-labeled antigen with a phage display library expressing a plurality of fragment antigen-binding (Fab) regions of an antibody;
allowing the plurality of barcode-labeled antigens to bind to the plurality of Fab regions of the phage to form an antibody-antigen binding complex comprising eukaryotic cells bound to phages;
separating the antibody-antigen binding complex into single-cell partitions (e.g., droplet-based or well-based assays);
introducing into each single-cell partitions a unique cell barcode-labeled bead;
preparing a single-cell cDNA library from the single-cell partitions;
performing PCR amplification reactions to produce a plurality of amplicons, wherein the amplicons comprise: 1) the cell barcode and the antigen barcode, 2) the cell barcode and a paired Fab sequence, and 3) a unique molecular identifier (UMI); and
sequencing the plurality of amplicons.

In some aspects, the eukaryotic cells are mammalian cells. In some aspects, the mammalian cells are HEK293T cells. In some aspects the mammalian cells are CHO cells. In some aspects, the eukaryotic cells are yeast cells. In some aspects, the yeast are Saccharomyces cerevisiae cells.

In some aspects, the barcode-labeled antigens are labeled with a first barcode comprising a DNA sequence or an RNA sequence.

In some aspects, the cell barcode-labeled beads are labeled with a second barcode comprising a DNA sequence or an RNA sequence.

In some aspects, the barcode-labeled antigens comprise an antigen from a pathogen or an animal. In some aspects, the animal is a human. In some aspects, the antigen from the animal comprises an antigen from a tumor-associated antigen or neoantigen. In some aspects, the antigen from a pathogen comprises an antigen from a nosocomial infection causing bacteria. In some aspects, the nosocomial infection causing bacteria comprises C. difficile, S. aureus, A. baumannii, or a combination thereof.

In some aspects, the plurality of antigens comprises a panel of epitope knock-outs. In some aspects, the plurality of antigens comprises a panel of antigen variants or mutations for epitope mapping.

In some aspects, the phage display expresses a plurality of natively or combinational paired single chain variable fragments (scFvs).

In some aspects, the method further includes determining a binding score by counting the number of UMIs for each paired Fab sequence.

Also disclosed herein is a method for simultaneous detection of an antigen and a protein.

In various aspects, the method includes:
contacting a population of eukaryotic cells engineered to express a plurality of barcode-labeled proteins with a yeast display library expressing a plurality of antigens;
allowing the plurality of barcode-labeled proteins to bind to the plurality of antigens of the phage to form a protein-antigen binding complex comprising eukaryotic cells bound to yeast cells;
separating the protein-antigen binding complex into single-cell partitions (e.g., droplet-based or well-based assays);
   introducing into each single-cell partitions a unique cell barcode-labeled bead;
   preparing a single-cell cDNA library from the single-cell partitions;
performing PCR amplification reactions to produce a plurality of amplicons, wherein the amplicons comprise: 1) the cell barcode and the protein barcode, 2) the cell barcode and an associated protein and antigen sequence, and 3) a unique molecular identifier (UMI); and
   sequencing the plurality of amplicons.

In some aspects, the eukaryotic cells are mammalian cells. In some aspects, the mammalian cells are HEK293T cells. In some aspects the mammalian cells are CHO cells. In some aspects, the eukaryotic cells are yeast cells. In some aspects, the yeast are Saccharomyces cerevisiae cells.

In some aspects, the barcode-labeled proteins are labeled with a first barcode comprising a DNA sequence or an RNA sequence.

In some aspects, the cell barcode-labeled beads are labeled with a second barcode comprising a DNA sequence or an RNA sequence.

In some aspects, the barcode-labeled proteins comprise a receptor proteins associated with viral infection. In some aspects, the receptor proteins comprise human receptor proteins. In some aspects, the human receptor proteins comprise proteins from human epithelial cells.

In some aspects, the method further includes determining a binding score by counting the number of UMIs for each paired Fab sequence.

In some aspects, also described herein are methods for simultaneous detection of an antigen and an antibody or binding fragment thereof that specifically binds said antigen. In various aspects, the method includes: contacting a population of eukaryotic cells engineered to display a plurality of barcode-labeled antigens with a population of antigen-specific B cells;
allowing the plurality of barcode-labeled antigens to bind to the population of antigen-specific B cells to form an antibody-antigen binding complex comprising eukaryotic cells bound to B cells;
separating the antibody-antigen binding complex into single-cell partitions (e.g., droplet-based or well-based assays);
introducing into each single-cell partitions a unique cell barcode-labeled bead;
preparing a single-cell cDNA library from the single-cell partitions;
performing PCR amplification reactions to produce a plurality of amplicons, wherein the amplicons comprise: 1) the cell barcode and the antigen barcode, 2) the cell barcode and a paired antibody or antibody binding fragment sequence, and 3) a unique molecular identifier (UMI); and
sequencing the plurality of amplicons.

In some aspects, the eukaryotic cells are mammalian cells. In some aspects, the mammalian cells are HEK293T cells. In some aspects the mammalian cells are CHO cells. In some aspects, the eukaryotic cells are yeast cells. In some aspects, the yeast are Saccharomyces cerevisiae cells.

In some aspects, the barcode-labeled antigens are labeled with a first barcode comprising a DNA sequence or an RNA sequence.

### DESCRIPTION OF DRAWINGS

The accompanying figures, which are incorporated in and constitute a part of this specification, illustrate aspects described below.

FIG. 1 shows a flow chart depicting an example approach for the simultaneous identification of antigens and antibodies according to the present disclosure.

FIGS. 2A-2C show (FIG. 2A) an illustrative examples of a custom mammalian display vector; (FIG. 2B) an example barcode sequence for mammalian display vector (SEQ ID NO. 167); and (FIG. 2C) mammalian surface display library generation (antigen/host protein).

FIG. 3 shows an overview of the generation of a scFv phage display library from PBMCs of a subject according to one aspect of the present disclosure.

FIG. 4 shows a method for transforming a barcoded antigen display plasmid library into yeast cells to form a yeast surface antigen display library.

FIGS. 5A-5D show overviews of (FIG. 5A) high-throughput simultaneous identification of antigens and antibodies using mammalian display library and phage display library screening approach; (FIG. 5B) high-throughput simultaneous identification of antigens and antibodies using mammalian display library and antigen-specific B cell screening approach; (FIG. 5C) high-throughput simultaneous identification of antigens and host proteins using mammalian display library and yeast display library screening approach; and (FIG. 5D) high-throughput simultaneous identification of host proteins and potently neutralizing antibodies against a pathogen via ligand blocking.

FIG. 6 shows an overview of the process for determining binding affinity between corresponding antigens and antibodies using a yeast antigen display library according to one aspect of the present disclosure.

### DETAILED DESCRIPTION

Disclosed herein are systems and methods for simultaneous detection of antigens and antigen specific antibodies or binding fragments thereof.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here can include the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

As used herein, the term “antigen” refers to a molecule that is capable of binding to an antibody. In some embodiment, the antigen stimulates an immune response such as by production of antibodies specific for the antigen. Antigens of the present disclosure can be, for example, an antigen from human immunodeficiency virus (HIV), an antigen from influenza virus, or an antigen from respiratory syncytial virus (RSV). Antigens of the present disclosure can also be, for example, a human antigen (e.g. VEGF, or an oncogene-encoded protein).

In the present disclosure, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to specifically interact with the HIV virus, such that the HIV viral infection is prevented, inhibited, reduced, or delayed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In human, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. The term “Fab region”, as used herein, refers to the region of an antibody composed of one constant and one variable domain from each heavy and light chain of the antibody, and which contains the sites involved in antigen binding. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M.J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

As used herein, the term “mammalian display library” refers to a library comprising polynucleotides encoding different recombinant polypeptides that can be displayed on mammalian cells. In some contexts, the term is also used generally to include mammalian cells transfected with the library of polynucleotides.

As used herein, the term “antigen-specific B cell” refers to a B cell that expresses antibodies that are able to distinguish between an antigen of interest and other antigens. The antigen specific B cell specifically bind to that antigen of interest with high or low affinity but which do not bind to other antigens.

### Systems and Methods

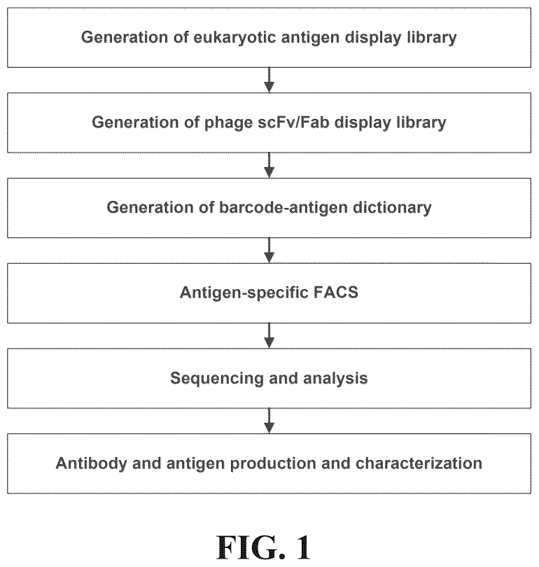

Disclosed herein are systems and methods for simultaneous detection of antigens and antigen specific antibodies or binding fragment thereof. FIG. 1 depicts a process overview of a method for simultaneous detection of antigens and antigen specific antibodies according to one aspect.

In some aspects, the method includes: contacting a population of eukaryotic cells engineered to display a plurality of barcode-labeled antigen with a phage display library expressing a plurality of fragment antigen-binding (Fab) regions of an antibody;
allowing the plurality of barcode-labeled antigens to bind to the plurality of Fab regions of the phage to form an antibody-antigen binding complex comprising eukaryotic cells bound to phages;
separating the antibody-antigen binding complex into single-cell partitions (e.g., droplet-based or well-based assays);
introducing into each single-cell partitions a unique cell barcode-labeled bead;
preparing a single-cell cDNA library from the single-cell partitions;
performing PCR amplification reactions to produce a plurality of amplicons, wherein the amplicons comprise: 1) the cell barcode and the antigen barcode, 2) the cell barcode and a paired Fab sequence, and 3) a unique molecular identifier (UMI); and
sequencing the plurality of amplicons.

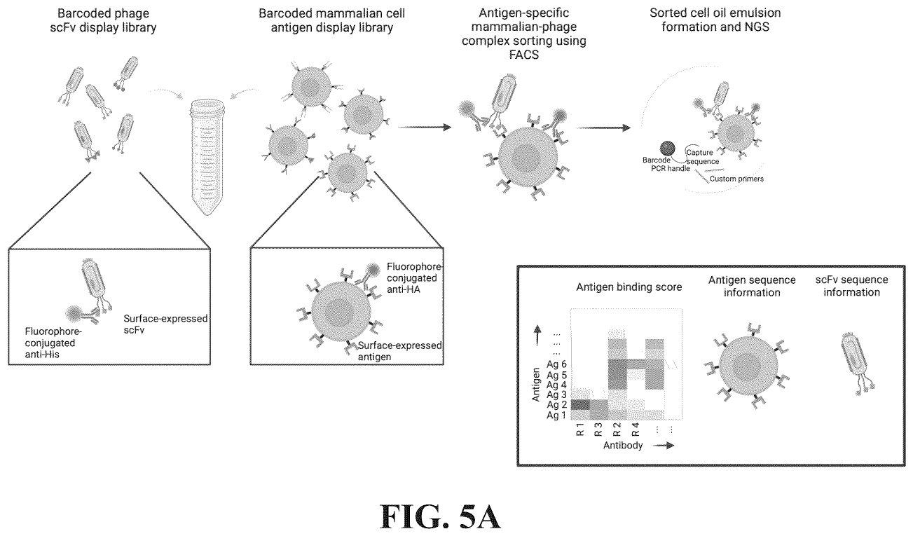

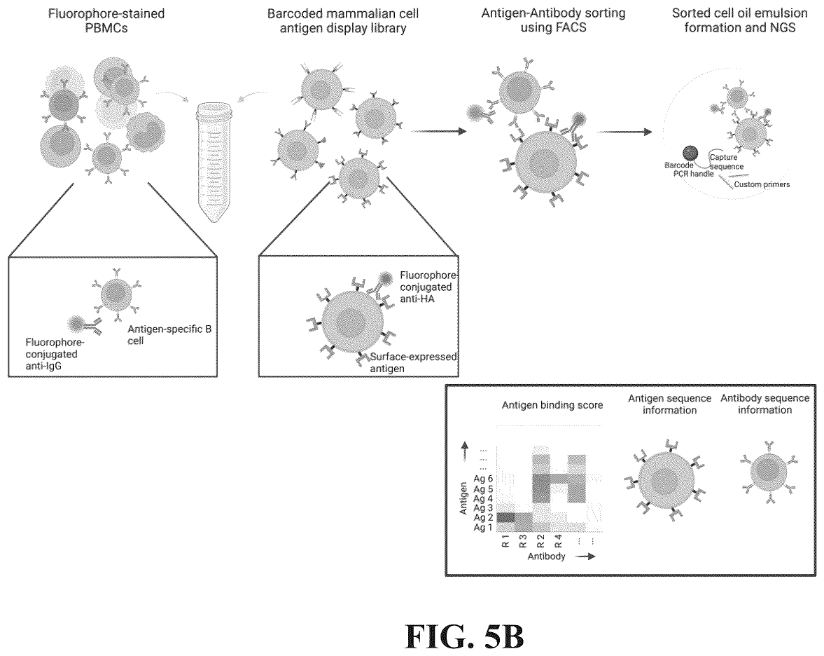

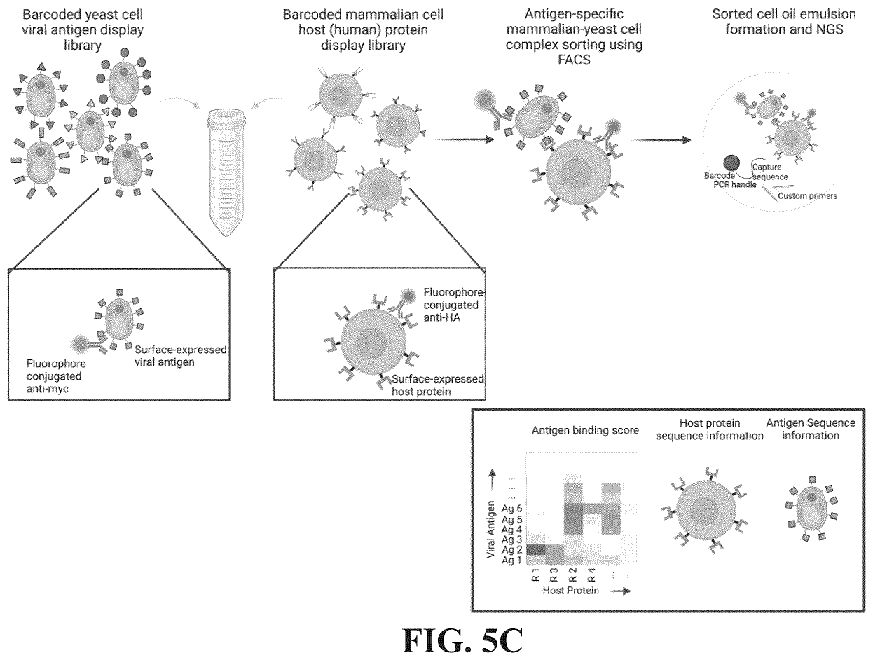

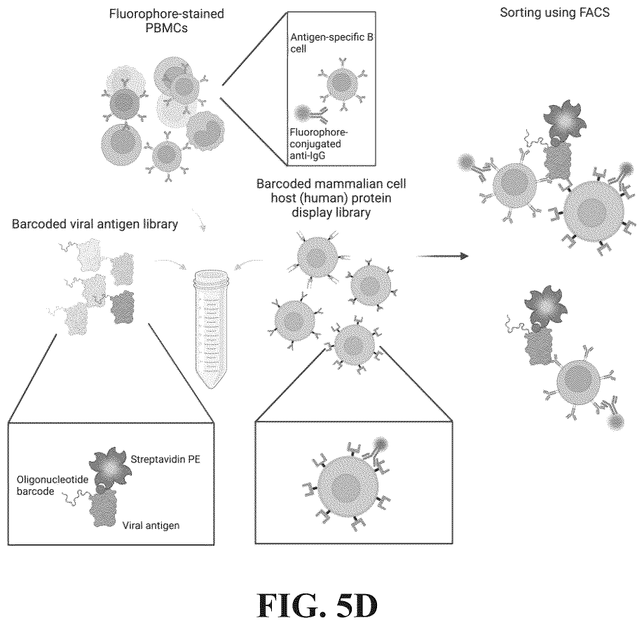

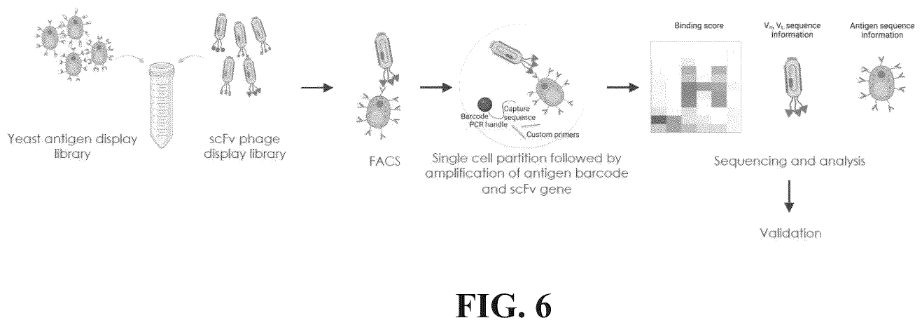

In some aspects, the eukaryotic cells are mammalian cells. The term “mammalian cells” means cells derived from mammalian species. A process overview showing the simultaneous detection of antigens and antigen specific antibodies or binding fragment thereof using a mammalian antigen display library is shown in FIGS. 5A-5D. Examples of suitable mammalian cells can include are human cells, feline cells, canine cells, bovine cells, equine cells, caprine cells, ovine cells, porcine cells, murine cells, such as cells from mice and rats, and cells from rabbits. In some aspects, the eukaryotic cells are mammalian cells. In some aspects, the mammalian cells are HEK293T cells. In some aspects the mammalian cells are CHO cells. In some aspects, the eukaryotic cells are yeast cells. In some aspects, the yeast are Saccharomyces cerevisiae cells. A process overview showing the simultaneous detection of antigens and antigen specific antibodies or binding fragment thereof using a yeast antigen display library is shown in FIG. 6.

It should be understood that the barcode described above is conjugated to the barcode-labeled antigen in a way that is known to one of ordinary skill in the art. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. An oligonucleotide barcode can also be conjugated to an antigen using the Solulink Protein-Oligonucleotide Conjugation Kit (TriLink cat no. S-9011) according to manufacturer's instructions. Briefly, the oligo and protein are desalted, and then the amino-oligo is modified with the 4FB crosslinker, and the biotinylated antigen protein is modified with S-HyNic. Then, the 4FB-oligo and the HyNic-antigen are mixed together. This causes a stable bond to form between the protein and the oligonucleotide.

As used herein, “beads” is not limited to a specific type of bead. Rather, a large number of beads are available and are known to one of ordinary skill in the art. A suitable bead may be selected on the basis of the desired end use and suitability for various protocols. In some embodiments, the bead is or comprises a particle or a bead. Beads can comprise particles that have been described in the art in, for example, U.S. Pat. Nos. 5,084,169, 5,079,155, 473,231, and 8,110,351. The particle or bead size can be optimized for binding a cell in a single cell emulsion and optimized for the subsequent PCR reaction.

In some embodiments, the barcode-labeled antigens are labeled with a first barcode comprising a DNA sequence or an RNA sequence. In some embodiments, the barcode-labeled antigens are labeled with a first barcode comprising a DNA sequence. In some embodiments, the barcode-labeled antigens are labeled with a first barcode comprising an RNA sequence.

In some aspects, the cell barcode-labeled beads are labeled with a second barcode comprising a DNA sequence or an RNA sequence. In some embodiments, the cell barcode-labeled beads are labeled with a second barcode comprising a DNA sequence. In some embodiments, the cell barcode-labeled beads are labeled with a second barcode comprising an RNA sequence. In some embodiments, the cell barcode-labeled beads are labeled with a barcode on the inside of the bead. In some embodiments, the cell barcode-labeled beads are labeled with a barcode encapsulated within the bead. In some embodiments, the cell barcode-labeled beads are labeled with a barcode on the outside of the bead.

These oligos, which contain the cell barcode, both: (1) enable amplification of cellular mRNA transcripts through the template switch oligo that is part of the oligo containing the cell barcode, and (2) directly anneal to the antigen barcode-containing oligos from the antigen. In some embodiments, the oligos delivered from the beads have the general structure: P5_PCR_handle-Cell_barcode-UMI-Template_switch_oligo.

In some aspects, the barcode-labeled antigens comprise an antigen from a pathogen or an animal. In some aspects, the animal is a mammal, including, but not limited to, primates (e.g., humans and nonhuman primates), cows, sheep, goats, horses, dogs, cats, rabbits, rats, mice and the like. In some aspects, the animal is a human. In some aspects, the antigen from the animal comprises an antigen from a tumor-associated antigen or neoantigen. “Neoantigens” as used herein generally include antigens that are present on the surface of cancer cells but are absent from the surface of normal cells of a patient. In some aspects, the antigen from a pathogen comprises an antigen from a nosocomial infection causing bacteria. In some aspects, the nosocomial infection causing bacteria comprises C. difficile, S. aureus, A. baumannii, or a combination thereof.

In some aspects, the plurality of antigens comprises a panel of epitope knock-outs. In some aspects, the plurality of antigens comprises a panel of antigen variants or mutations for epitope mapping. For example, a mutant display library of antigens can be formed by introducing point mutations in residues of an antigen of interest before cloning said constructs into a mammalian surface display vector (e.g., via gateway cloning).

In some aspects, the phage display expresses a plurality of natively or combinational paired single chain variable fragments (scFvs). The Fab regions can include antibodies or fragments thereof obtained from a population of peripheral blood mononuclear cells (PBMCs) of a subject. The term “peripheral blood mononuclear cells” or “PBMCs” as used herein refers to cells with a single nucleus in peripheral blood, including various lymphocyte and monocyte. Examples of peripheral blood mononuclear cells include, but are not limited to, T or B lymphocytes, monocytes, NK cells, dendritic cells, precursors thereof and committed and non-committed hematopoietic stem cells.

In some aspects, the method further includes determining a binding score by counting the number of UMIs for each paired Fab sequence.

A number of single cell workflows can be utilized in the present systems and methods for the simultaneous detection of antigens and antigen specific antibodies or binding fragment thereof. For example, the systems and methods can use microwell arrays or microwell cartridges (e.g., BD Rhapsody™) or microfluidics devices (e.g., 10× Genomics (San Francisco, Calif.), Drop-seq (McCarroll Lab, Harvard Medical School (Cambridge, Mass.); Macosko et al., Cell, 2015 May 21 16; 5:1202, the content of which is incorporated herein by reference in its entirety), or Abseq (Mission Bio (San Francisco, Calif.); Shahi et al., Sci Rep. 2017 Mar. 14; 7:44447, the content of which is hereby incorporated by reference in its entirety) in combination with solid or semisolid particles associated with barcodes, such as stochastic barcodes (e.g., BD Rhapsody, or Drop-seq), or disruptable hydrogel particles enclosing releasable barcodes, such as stochastic barcodes (e.g., 10× Genomics, or Abseq). Single cell partitions, such as, for example, microwell cartridges (e.g., BD Rhapsody™) comprising unique barcode sequences in each partition (e.g., well) can enable a user to associate cell labels from sequencing data with a particular partition. Examples suitable workflows include those discussed in U.S. Pat. App. Pub. No. 2021/0302422A1, which is hereby incorporated by reference in its entirety.

Also disclosed herein is a method for simultaneous detection of an antigen and a protein or fragment thereof that specifically binds said antigen. In various aspects, the method includes:
contacting a population of eukaryotic cells engineered to express a plurality of barcode-labeled proteins with a phage display library expressing a plurality of antigens;
allowing the plurality of barcode-labeled proteins to bind to the plurality of antigens of the phage to form a protein-antigen binding complex comprising eukaryotic cells bound to phages;
separating the protein-antigen binding complex into single-cell partitions (e.g., droplet-based or well-based assays);
   introducing into each single-cell partitions a unique cell barcode-labeled bead;
   preparing a single-cell cDNA library from the single-cell partitions;
performing PCR amplification reactions to produce a plurality of amplicons, wherein the amplicons comprise: 1) the cell barcode and the protein barcode, 2) the cell barcode and an associated protein and antigen sequence, and 3) a unique molecular identifier (UMI); and
   sequencing the plurality of amplicons.

In some aspects, the eukaryotic cells are mammalian cells. The term “mammalian cells” means cells derived from mammalian species. Examples of suitable mammalian cells can include are human cells, feline cells, canine cells, bovine cells, equine cells, caprine cells, ovine cells, porcine cells, murine cells, such as cells from mice and rats, and cells from rabbits. In some aspects, the eukaryotic cells are mammalian cells. In some aspects, the mammalian cells are HEK293T cells. In some aspects the mammalian cells are CHO cells. In some aspects, the eukaryotic cells are yeast cells. In some aspects, the yeast are Saccharomyces cerevisiae cells.

In some aspects, the barcode-labeled antigens are labeled with a first barcode comprising a DNA sequence or an RNA sequence. In some embodiments, the barcode-labeled antigens are labeled with a first barcode comprising a DNA sequence. In some embodiments, the barcode-labeled antigens are labeled with a first barcode comprising an RNA sequence.

In some aspects, the cell barcode-labeled beads are labeled with a second barcode comprising a DNA sequence or an RNA sequence. In some embodiments, the cell barcode-labeled beads are labeled with a second barcode comprising a DNA sequence. In some embodiments, the cell barcode-labeled beads are labeled with a second barcode comprising an RNA sequence. In some embodiments, the cell barcode-labeled beads are labeled with a barcode on the inside of the bead. In some embodiments, the cell barcode-labeled beads are labeled with a barcode encapsulated within the bead. In some embodiments, the cell barcode-labeled beads are labeled with a barcode on the outside of the bead.

These oligos, which contain the cell barcode, both: (1) enable amplification of cellular mRNA transcripts through the template switch oligo that is part of the oligo containing the cell barcode, and (2) directly anneal to the antigen barcode-containing oligos from the antigen. In some embodiments, the oligos delivered from the beads have the general structure: P5_PCR_handle-Cell_barcode-UMI-Template_switch_oligo.

In some aspects, the barcode-labeled proteins comprise a receptor proteins associated with viral infection. In some aspects, the receptor proteins comprise human receptor proteins. As used herein, the term “receptor proteins associated with viral infections” refers to a specific molecular component of the cell, which is capable of recognizing and interacting with a virus, and which, after binding to said virus, is capable of generating a signal that initiates a chain of events leading to a biological response. The receptor proteins do not necessarily include a full-length sequence and can refer to a fragment thereof. Such receptor protein may further be a variant sequence formed by amino acid substitution, deletion, or addition or part of a receptor protein, provided its recognition and/or interaction with the viral pathogen is substantially maintained. In some aspects, the human receptor proteins comprise proteins from human epithelial cells.

In some aspects, the method further includes determining a binding score by counting the number of UMIs for each paired Fab sequence.

The current technology provides a number of improvements and benefits over previous technologies. For example, the antigen protein of interest can be tagged with an arbitrary barcode and sequence as an identifier. In addition, the simple presence or absence of a cell barcode is generally not sufficient to identify antigen specificity. Thus, in some embodiments, in addition to a cell barcode, there is also a unique molecular identifier (UMI) on each bead-delivered oligo. Thus, the specificity can be determined based on the number of UMIs per cell barcode: antigen barcode pair.

### EXAMPLES

The following examples are set forth below to illustrate the systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. High-Throughput Simultaneous Identification of Antigen-Antibody Pairs by Screening scFv/Fab Phage Display Library Using Mammalian Antigen Display Library

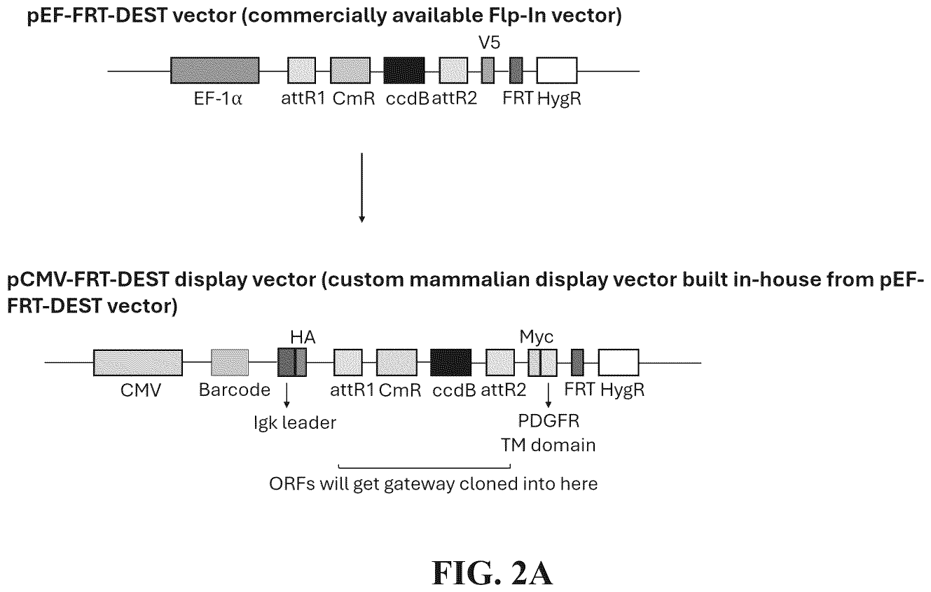

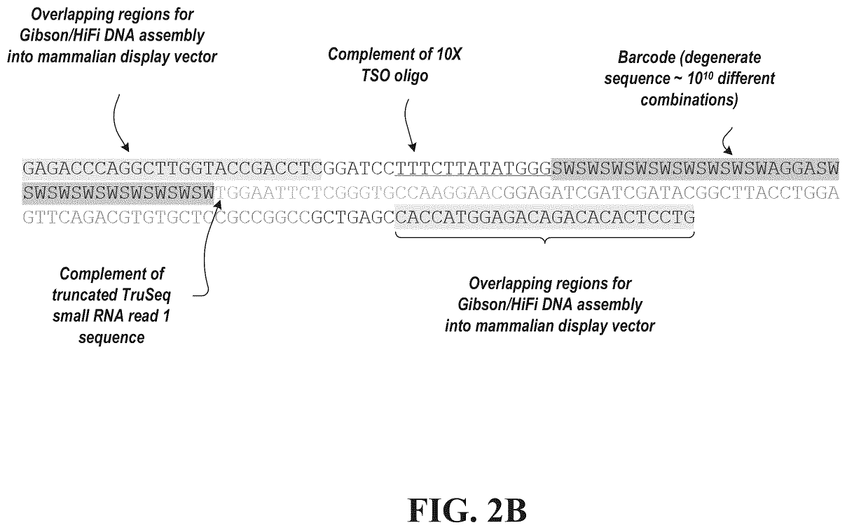

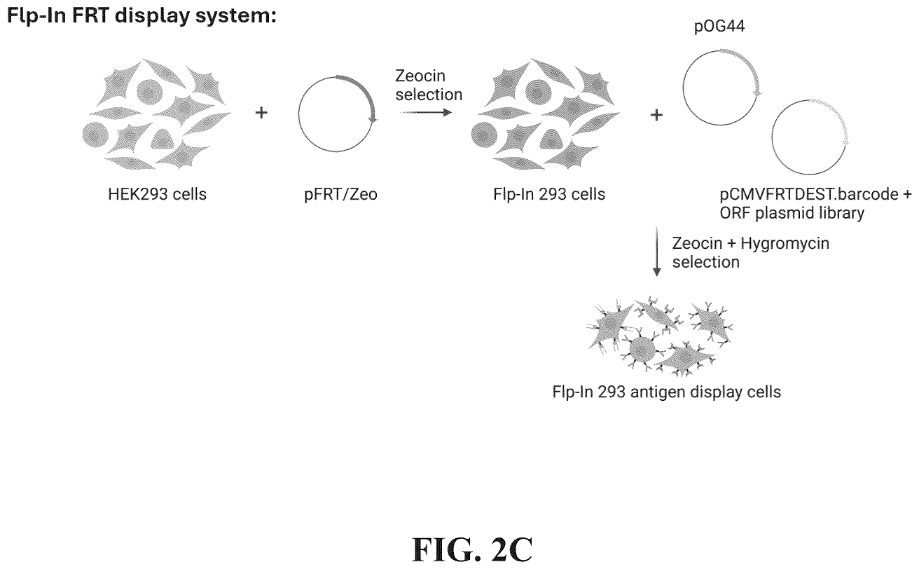

Mammalian barcoded antigen display library generation. The first step for the generation of a barcoded antigen display library is the generation of a plasmid library encoding peptide or full-length protein associated with a unique barcode sequencing. FIGS. 2A-2C depict an overview of the formation of mammalian barcoded antigen display libraries. The generation of such a plasmid library enables the expression of tens of thousands of peptides or proteins in a single reaction including proteins that may be difficult to express recombinantly otherwise. The plasmid library is generated using gateway cloning. Gateway cloning involves the construction of two plasmids—entry clone library and destination vector. The open reading frame (ORF) encoding peptides or full-length proteins is cloned into a pDONR™ vector for the construction of the entry clone library. The entry clone library is generated via a BP reaction involving recombinant sites, attB1 and attB2 in the ORFs and recombinantion sites, attP1 and attP2 in the entry vector using Gateway™ BP clonase™ II enzyme (Invitrogen). The entry clone library has the recombination sites attL1 and attL2. The entry clone library construction can be used, for example, in the high-throughput identification of new tumor-associated antigens and neoantigens: total RNA will be extracted from early-stage cancer cells/tissues and the mRNA will be reverse transcribed into cDNA and cloned into pDONR™ vector (example, pDONR222 vector) using the CloneMiner II cDNA library construction kit (Invitrogen). In other examples, the entry clone library is used for the identification of antigen-antibody pairs associated with nosocomial bacterial infections: Total RNA is extracted from cultures of nosocomial infection causing bacteria such as C. difficile, S. aureus and A. baumannii using the RNAqueous™ total RNA isolation kit (Invitrogen) and enriched for mRNA from the total RNA using the MICROBExpress™ bacterial mRNA enrichment kit (Invitrogen). Poly(A) tail will then be added to the bacterial mRNA using E. coli poly(A) polymerase enzyme. The poly (A) tailed mRNA will be reverse transcribed into cDNA and cloned into pDONR™ vector using the CloneMiner II cDNA library construction kit (Invitrogen). In yet another example, the entry clone is used for the identification of antigen-antibody pairs associated with HIV-1: synthesized genes corresponding to HIV-1 envelope peptides that have been shown to elicit strong immune response will be synthesized with attB1 and attB2 sites and cloned into the pDONR™ vector.

The ORFs from the entry clone library are cloned into the destination vector via a LR reaction involving recombination sites attL1 and attL2 in the former and recombination sites attR1 and attR2 in the latter using Gateway™ LR clonase™ II enzyme (Invitrogen). Prior to the LR reaction, a custom destination vector/mammalian display vector (FIG. 2A) is constructed using a Flp-In gateway destination vector as the plasmid libraries will be stably transfected into Flp-In™ cell lines (such as the Flp-In™ 293 cells) for expression of a single type of protein per cell. The custom destination vector is constructed by replacing the EF-1α promoter with CMV promoter. A secretion signal (such as Igk leader sequence) and a transmembrane domain (such as transmembrane domain sequence of PDGFR) are inserted on the N- and C-terminus of attR1 and attR2 sites. The V5 epitope tag on the vector is replaced with a HA tag downstream from the secretion signal and a Myc tag upstream to the transmembrane domain. The HA and Myc tags are included for detection of protein/peptide surface expression on mammalian cells using FACS. Following gateway cloning of the ORFs into the custom destination vector, an oligonucleotide pool consisting of a 40 nucleotide long degenerate barcode sequence (for example, (SW)₁₈-AGGA-(SW)₁₈, where S=G/C; W=A/T), complement of 10× template switching oligo (TSO) sequence and complement of truncated Truseq small RNA read 1 sequence (FIG. 2B) amplified using polymerase chain reaction (PCR) is cloned into the destination vector via Gibson/HiFi DNA assembly, downstream the CMV promoter sequence. The degeneracy of the oligonucleotide pool can allow for the addition of unique barcodes to the ORFs. The addition of unique barcodes this way can overcome the need for a separate barcode conjugation step of the expression peptides or proteins. The use of a separate barcode conjugation step usually requires a huge amount of recombinantly expressed peptides or proteins while having a poor recovery rate. Cloning of the ORFs can also be performed by conventional restriction enzyme digestion and ligation or using Gibson/HiFi DNA assembly into the mammalian display expression vectors following the addition of the appropriate flanking sequences. Following cloning of the ORFs, the LR reaction of ligation mix is transformed into E. coli electrocompetent cells (such as Electromax DH10B cells) to obtain the cell surface display plasmid library containing ˜10⁶ independent clones.

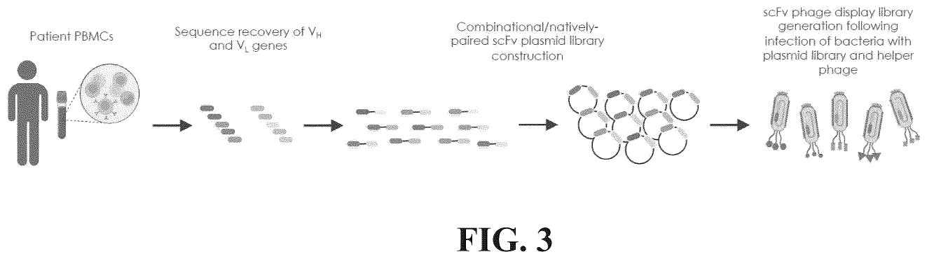

scFv/Fab phage display library generation. For scFv (single chain variable fragment) phage display library construction, total RNA is extracted from patient PBMCs (for example, patients with early-stage cancer, patients with past or current infection, or from healthy donors). An overview of the generation of an scFv phage display library from patient PBMCs is shown in FIG. 3. The total RNA is then reverse transcribed into cDNA and subject to PCR to specifically amplify the variable heavy and light chain domains of the B cell receptors (BCRs) and introduce overlapping nucleotide regions for Gibson assembly of the variable heavy and light chains into a phagemid vector (example, pComb3XSS, pAK100) as well as to introduce a linker (example, (G₄S)₃, (G₄S)₄) between the variable heavy and light chains. For vectors that may not have a His tag in their backbone, a His tag is cloned downstream and in-frame to the scFv fragments. The resulting phagemids with scFv are then transformed into electro-competent E. coli cells (for example, TG1 cells) and plated on 2× YT, 2% glucose agar plates with the appropriate antibiotic. The next day, colonies are scraped off using 2× YT media containing glycerol and stored at −80° C. For phage rescue, the glycerol stock is grown to log-phage and infected with 10 times excess of helper phage (example, M13K07). The infected cells are then grown overnight in 2× YT media with appropriate antibiotics for amplification of scFv-displaying phage. The overnight cultures are centrifuged, and the supernatant is collected and mixed with PEG6000/NaCl solution to precipitate phage. The precipitated phage is pelleted and washed with ice-cold DPBS. The washed phage is re-precipitated. The resulting pelleted phage is resuspended in ice-cold DPBS, and centrifuged once more to remove any residual bacteria, and the supernatant is saved as the rescued phage. For Fab (fragment antigen-binding) phage display library constructions, the total RNA is extracted from patient PBMCs or from healthy donors and is reverse transcribed into cDNA. From the cDNA, the heavy chain Fd and κ light chain (and/or λ light chain) are specifically amplified and assembled into a phagemid vector. For vectors that may not have a His tag in their backbone, a His tag is cloned downstream and in-frame to the Fab fragments. Transformation into electrocompetent E. coli cells, Fab-expressing phage amplification, and phage rescue are performed as described above.

Antigen-barcode dictionary generation. Following the construction of the plasmid library for mammalian display, an antigen-barcode dictionary to identify the barcode associated with each ORF is constructed using Oxford Nanopore long read sequencing approach. The long read sequencing approach can allow for the sequencing of the whole length of the plasmid libraries without the need for assembly. The plasmid library will be prepared for long read sequencing using the Rapid Sequencing kit V14 and loaded onto a R10.4.1 flow cell for sequencing using Nanopore MinION Mk1C sequencer. Basecalling of the generated sequences will be performed using a high accuracy model and subject to bioinformatic analysis using python and R for the construction of the antigen-barcode dictionary. The dictionaries may also be constructed using PacBio or Illumina sequencing when appropriate handles or adapter sequences are added to the oligonucleotide pool consisting of the degenerate barcode sequence.

Antigen-specific FACS. The plasmid library is stably transfected into mammalian cells (such as Flp-In™ 293 cells) for surface expression of a single type of protein per cell. Also, by using a stable transfection system, it is possible to eliminate the need for new transfections each time and the cells following transfection and storage can be revived for future tests or repeated analyses. The stable transfection of the mammalian cells can be achieved using transfection agents such as Lipofectamine 2000 or calcium phosphate. Successful transfection and expression of the antigens can be assessed using flow cytometry. The rescued phage is then mixed and incubated with the mammalian display library in a rotator at 4° C. Following incubation, the cells are stained with a fluorophore-conjugated antibody (such as FITC-conjugated anti-HA antibody) to the HA tag on the antigens on the mammalian cell surface and a fluorophore-conjugated antibody (such as APC anti-His tag antibody) to the His tag on the scFv/Fab expressed on phage. To collect mammalian cells bound to phage via antigen-antibody binding, cells that are live and positive for the two fluorophores, one on the anti-HA tag antibody and the other on the anti-His tag antibody are sorted, resuspended in DMEM media to 800-1200 cells/μL at a viability of >90%. The sorted cells can then be subject to subsequent library preparation, sequencing, and analysis.

To sort memory B cells expressing antigen-specific antibodies from patient/healthy PBMCs that may bind the antigens on the barcoded mammalian display library using FACS, the PBMCs will be stained with a panel of fluorophore-conjugated antibodies specific to cell markers on T cells (CD3), monocytes (CD14) and B cells (CD19, IgG, IgM). The B cells that are live, CD3⁻, CD14⁻, CD19⁺, IgM⁻, IgG⁺ bound to antigen-displaying mammalian cells will be bulk sorted and resuspended in complete RPMI to 800-1200 cells//μL at a viability >90%.

Sequencing and analysis. The sorted cells can then be processed for library preparation using single-cell analysis systems such as 10× Genomics, BD Rhapsody, or others. For library preparation, the sorted cells are partitioned into single-cell suspensions using microfluidic circuits/microwells as per the manufacturer's protocol. The protocol will include a minor modification for the amplification and purification of antigen barcode libraries and scFv/Fab libraries/BCR libraries. Briefly, the sorted cells are partitioned into individual cells prior to lysis. Upon lysis, the mRNA from the mammalian cells and memory B cells (if sorting B cells directly) is reverse transcribed into cDNA and the Next Generation Sequencing (NGS) oligo adaptor present on the beads can capture the cDNA and the V_{L}-V_{H} gene from scFv/Fab phagemid/cDNA from memory B cells and add a unique barcode and unique molecular identifiers (UMI) that can allow for the downstream association of the antigen with the scFv/BCR sequence. Alternatively, polyadenylated sequences on mRNAs and scFv/Fab phagemid (phagemids with cloned polyadenylated sequences) are captured by the beads, after which cDNA synthesis is performed. The cellular cDNA from the mammalian cells and the phagemid/memory B cells are amplified using custom primers that flank the cDNA antigen barcodes and the V(D)J genes of BCR/scFv/Fab genes. The resulting amplified libraries are purified using SPRI beads (1.6× purification), and the BCR/scFv/Fab genes and antigen barcode libraries are sequenced using sequencers such as NovaSeq 6000 with a target of 10000 reads or more per cell. Output fastq files are processed to assemble, quantify, and annotate paired BCR/scFv/Fab sequences and antigen barcodes on a cell-by-cell basis using barcodes/UMI introduced via the beads. The antigen barcode sequences associated with each single-cell are cross-referenced with the antigen-barcode dictionary to simultaneously discover the antigen that binds a BCR/scFv/Fab. The amplified libraries may also be sequenced using long read sequencing approaches following end repair and appropriate adapter ligation.

Binding score determination. To determine the specificity of a BCR/scFv/Fab to its associated antigen, each BCR/scFv/Fab binding an antigen is given a binding score. The binding score is calculated by counting the number of UMIs for the respective BCR/scFv/Fab and calculating the centered-log ration (CLR) of each BCR/scFv/Fab UMI for each cell. All counts of 1, 2, and 3 UMI are set to 0 to account for background noise.

Validation and/or characterization of new antigens and antibodies. Antigens and BCR/scFv/Fab to those antigens with a binding score >1 are shortlisted. The variable heavy and light chain sequences of the shortlisted BCR/scFv/Fab are cloned into expression vectors with constant heavy and light chain sequences and co-transfected into mammalian Expi293F cells using a transfection reagent (example, Expifectamine) for micro-expression of IgG. The antigen sequences can also be cloned into expression vectors (for example, pcDNA3.1(+)) and transfected into mammalian Expi293F cells for expression. The recombinantly expressed antigens are purified using affinity chromatography and size exclusion chromatography. The expressed IgGs in supernatants can then be tested for binding to antigens using ELISA. To perform ELISA, the purified antigens are immobilized at a concentration of 2 μg/ml on Nunc immune 96 well plates. Following immobilization, the wells are blocked and incubated with the supernatants containing antibodies. The wells are washed, and the antigen-antibody binding is detected using HRP (horseradish peroxidase)-conjugated anti-human IgG secondary antibody. Data is collected at an absorbance of 450 nm using a plate reader following the addition of the TMB (3,3′,5,5′-Tetramethylbenzidine) substrate.

### Example 2. High-Throughput Simultaneous Identification of Host (Human) Receptor/Protein Associated with Pathogen Invasion/Infection by Mammalian Human Receptor/Protein Display Library Using Antigen Yeast Display Library or Recombinantly Expressed Antigens

Mammalian barcoded host (human) protein display library generation. The first step for the generation of a barcoded host protein display library is the generation of a plasmid library encoding proteins associated with a unique barcode sequencing. FIGS. 2A-2C depict an overview of the formation of mammalian barcoded host protein display libraries. The generation of such a plasmid library enables the expression of tens of thousands of proteins in a single reaction including proteins that may be difficult to express recombinantly otherwise. The plasmid library is generated using gateway cloning. Gateway cloning involves the construction of two plasmids—entry clone library and destination vector. The open reading frame (ORF) encoding peptides or full-length proteins is cloned into a pDONR™ vector for the construction of the entry clone library. The entry clone library is generated via a BP reaction involving recombinant sites, attB1 and attB2 in the ORFs and recombinantion sites, attP1 and attP2 in the entry vector using Gateway™ BP clonase™ II enzyme (Invitrogen). The entry clone library has the recombinantion sites attL1 and attL2. The entry clone library construction can be used, for example, in the high-throughput identification of host (human) receptors associated with pathogen invasion/infection: cDNA synthesized from total RNA extracted from cultured human mucosal epithelia that form the entry point for most pathogenic organisms, especially viruses is into pDONR™ vector (example, pDONR222 vector) using the CloneMiner II cDNA library construction kit (Invitrogen). The entry clone library can also be the Gateway cloning-adapted CCSB human ORFeome collection. The human ORFeome collection created by the Center for Cancer systems Biology of the Dana-Farber Institute represents more than 12,000 unique genes (including genes coding for cell surface receptors).

The ORFs from the entry clone library are cloned into the destination vector via a LR reaction involving recombination sites attL1 and attL2 in the former and recombination sites attR1 and attR2 in the latter using Gateway™ LR clonase™ II enzyme (Invitrogen). Prior to the LR reaction, a custom destination vector is constructed using a Flp-In gateway destination vector as the plasmid libraries will be stably transfected into Flp-In™ cell lines (such as the Flp-In™ 293 cells) for expression of a single type of protein per cell. The custom destination vector is constructed by replacing the EF-1α promoter with CMV promoter. A secretion signal (such as Igk leader sequence) and a transmembrane domain (such as transmembrane domain sequence of PDGFR) are inserted on the N- and C-terminus of attR1 and attR2 sites. The V5 epitope tag on the vector is replaced with a HA tag downstream from the secretion signal and a Myc tag upstream to the transmembrane domain. The HA and Myc tags are included for detection of protein/peptide surface expression on mammalian cells using FACS. Following gateway cloning of the ORFs into the custom destination vector, an oligonucleotide pool consisting of a 40 nucleotide long degenerate barcode sequence (for example, (SW)₁₈-AGGA-(SW)₁₈, where S=G/C; W=A/T), complement of 10× template switching oligo (TSO) sequence and complement of truncated Truseq small RNA read 1 sequence, amplified using polymerase chain reaction (PCR) is cloned into the destination vector via HiFi/Gibson DNA assembly, downstream the CMV promoter sequence. The degeneracy of the oligonucleotide pool can allow for the addition of unique barcodes to the ORFs. The addition of unique barcodes this way can overcome the need for a separate barcode conjugation step of the expression peptides or proteins. The use of a separate barcode conjugation step usually requires a huge amount of recombinantly expressed peptides or proteins while having a poor recovery rate. Cloning of the ORFs can also be performed by conventional restriction enzyme digestion and ligation or using Gibson/HiFi DNA assembly into the mammalian display expression vectors following the addition of the appropriate flanking sequences. Following cloning of the ORFs, the LR reaction of ligation mix is transformed into E. coli electrocompetent cells (such as Electromax DH10B cells) to obtain the cell surface display plasmid library containing ˜10⁶ independent clones.

Host protein-Barcode dictionary generation. Following the construction of the plasmid library for mammalian display, a host protein-barcode dictionary to identify the barcode associated with each ORF is constructed using Oxford Nanopore long read sequencing approach. The long read sequencing approach can allow for the sequencing of the whole length of the plasmid libraries without the need for assembly. The plasmid library will be prepared for long read sequencing using the Rapid Sequencing kit V14 and loaded onto a R10.4.1 flow cell for sequencing using Nanopore MinION Mk1C sequencer. Basecalling of the generated sequences will be performed using a high accuracy model and subject to bioinformatic analysis using python and R for the construction of the host protein-barcode dictionary. The dictionaries may also be constructed using PacBio or Illumina sequencing when appropriate handles or adapter sequences are added to the oligonucleotide pool consisting of the degenerate barcode sequence.

Barcoded Antigen yeast display library generation. For the generation of a barcoded antigen yeast surface display library, an oligonucleotide pool with degenerate barcode sequence (e.g., WSWSWSWSWSWSWSWSWSAGGAWSWSWSWSWSWSWSWSWS, where W=A/T, S=G/C), complement of 10× template switching oligo (TSO) sequence and complement of truncated Truseq small RNA read 1 sequence will be amplified using polymerase chain reaction (PCR) and assembled into the yeast surface display vector such as pETcon(−) using Gibson assembly. Prior to insertion of the barcode, attR1 and attR2 sites for gateway cloning of antigen genes (for example, viral proteins) will be inserted into the vector. Following gateway cloning of the antigen genes, the barcodes is cloned into the plasmid library via Gibson/HiFi DNA assembly. An antigen-barcode dictionary like the host protein-barcode dictionary will be generated. The barcoded yeast surface antigen display plasmid library are transformed into yeast cells such as S. cerevisiae EBY100 and induced for surface expression of the antigens in galactose-rich selective media. The antigens will be expressed as C-terminal fusion proteins to the anchor yeast protein Aga2p. An epitope tag such as a His tag on the fusion proteins will allow detection of surface expression in FACS using a fluorophore conjugated anti-His antibody.

Barcoded antigen library can also be prepared by recombinant expression. Plasmids encoding Avi-tagged antigens is transiently transfected into Expi293F cells using Gibco™ ExpiFectamine™ 293 transfection kit for expression. The constructs will have a His epitope tag for detection downstream. The expressed antigens are purified from the cell culture supernatants using affinity chromatography and size exclusion chromatography. The purified Avi-tagged antigens are biotinylated using BirA enzyme. Streptavidin-PE linked to a unique oligonucleotide/DNA barcode are conjugated to the antigens via biotin.

Antigen-specific FACS. The barcoded host protein plasmid library is stably transfected into mammalian cells (such as Flp-In™ 293 cells) for surface expression of a single type of protein per cell. Also, by using a stable transfection system, it is possible to eliminate the need for new transfections each time and the cells following transfection and storage can be revived for future tests or repeated analyses. The stable transfection of the mammalian cells can be achieved using transfection agents such as Lipofectamine 2000 or calcium phosphate. Successful transfection and expression of the host proteins can be assessed using flow cytometry. The antigen-expressing yeast cells/barcoded recombinantly expressed antigens are then mixed and incubated with the mammalian display library in a rotator at 4° C. Following incubation, the cells are stained with a fluorophore-conjugated antibody (such as FITC-conjugated anti-HA antibody) to the HA tag on the host proteins on the mammalian cell surface and a fluorophore-conjugated antibody (such as APC anti-His tag antibody) to the His tag on the yeast cells (in the case of recombinantly expressed antigens, they are already conjugated PE). To collect mammalian cells bound to yeast cells/recombinant antigen via host protein-antigen binding, cells that are live and positive for the two fluorophores, one on the anti-HA tag antibody and the other on the anti-His tag antibody are sorted, resuspended in DMEM media to 800-1200 cells/μL at a viability of >90%. The sorted cells can then be subject to subsequent library preparation, sequencing, and analysis.

For high-throughput simultaneous identification of host (human) receptors and potently neutralizing antibodies against the pathogen via ligand blocking (FIG. 5D), fluorophore-stained PBMCs from patients with active infection and/or healthy individuals previously exposed to the pathogen is mixed with the barcoded antigen library and the barcoded host protein library. Memory B cells (CD3⁻, CD14⁻, CD19⁺, IgM⁻, IgG⁺) bound to the antigens as well as to host (human) protein-antigen complexes are sorted. The rationale for sorting the two populations is that when an antibody binds to the antigen alone but not to the complex, the antibody might be binding to an epitope that prevents the interaction between the antigen and the host protein.

Sequencing and analysis. The sorted cells can then be processed for library preparation using single-cell analysis systems such as 10× Genomics, BD Rhapsody, or others. For library preparation, the sorted cells are partitioned into single-cell suspensions using microfluidic circuits/microwells as per the manufacturer's protocol. The protocol will include a minor modification for the amplification and purification of human protein barcode libraries and antigen yeast libraries/recombinantly expressed and barcoded antigens. Briefly, the sorted cells are partitioned into individual cells prior to lysis. Zymolase is also added for lysis of yeast cells. Upon lysis, the mRNA from the mammalian cells is reverse transcribed into cDNA and the Next Generation Sequencing (NGS) oligo adaptor present on the beads can capture the cDNA and yeast cell cDNA/oligonucleotide barcode on the recombinantly expressed antigens and add a unique barcode and unique molecular identifiers (UMI) that can allow for the downstream association of human protein with antigen sequence. Alternatively, polyadenylated sequences on mRNAs are captured by the beads, after which cDNA synthesis is performed. The cellular cDNA from the mammalian cells and the yeast cells/oligonucleotide barcodes on recombinant antigens are amplified using custom primers that flank the cDNA human protein barcodes and antigen barcodes/oligonucleotide barcodes. The resulting amplified libraries are purified using SPRI beads (1.6× purification), and the antigens barcodes/oligonucleotide barcodes and human protein barcode libraries are sequenced using sequencers such as NovaSeq 6000 with a target of at least 10000 reads per cell. Output fastq files are processed to assemble, quantify, and annotate paired antigen sequences and human protein barcodes on a cell-by-cell basis using the barcodes/UMI introduced from the beads. The human protein barcode sequences associated with each single-cell are cross-referenced with the human protein-barcode dictionary to simultaneously discover the human protein the antigens can bind. The amplified libraries may also be sequenced using long read sequencing approaches following end repair and appropriate adapter ligation. For high-throughput simultaneous identification of host (human) receptors and potently neutralizing antibodies against the pathogen via ligand blocking, the cellular cDNA from B cells will be amplified using primers that flank the V(D)J genes in the B cell receptor (BCR). The oligonucleotide with the antigen barcode and cDNA with the host protein barcode will be amplified using custom primers. Following purification and sequencing of the antibodies, antigen and host protein barcodes, the output fastq files will be processed using Cell Ranger to assemble, quantify and annotate the barcode sequences on a cell-by-cell basis.

Binding score determination. To design the specificity of an antigen to its associated human receptor, each antigen binding to a human protein is given a binding score. The binding score is calculated by counting the number of UMIs for the respective antigen and calculating the centered-log ratio (CLR) of each antigen UMI for each cell. All counts of 1, 2, and 3 UMI are set to 0 to account for background noise. With regards to high-throughput simultaneous identification of host (human) receptors and potently neutralizing antibodies against the pathogen via ligand blocking, for each antibody, antigen barcode UMI count matrix and host protein barcode UMI count matrix is be created and CLR is calculated. This allows the identification of antigen-host receptor pairs, along with both antibodies that are targeting epitopes outside of the respective receptor/protein binding site, as well as antibodies that can block the antigens from interacting with the respective host proteins.

Validation and/or characterization of antigens that may bind human proteins. Antigens and human proteins binding with a score >1 is shortlisted. The antigen sequences (as gBlock gene fragments, for instance) are cloned into expression vectors (example, pcDNA3.1(+)) and transfected into mammalian Expi293F cells using a transfection reagent (example, Expifectamine) for recombinant expression of antigens. In a similar way, host (human) proteins can also be recombinantly expressed. The recombinantly expressed antigens and human proteins are purified using affinity chromatography and size exclusion chromatography. The expressed antigens can then be tested for binding to human proteins using ELISA. To perform ELISA, the purified antigens are immobilized at a concentration of 2 μg/ml on Nunc immune 96 well plates. Following immobilization, the wells are blocked and incubated with the human proteins. The wells are washed, and the antigen-human protein binding is detected using an anti-tag antibody (such as anti-His, based on the tag on the human protein) conjugated to HRP. Data is collected at an absorbance of 450 nm using a plate reader following the addition of the TMB substrate. With regards to high-throughput simultaneous identification of host (human) receptors and potently neutralizing antibodies against the pathogen via ligand blocking, the variable heavy and light chain sequences of the shortlisted antibodies is cloned into expression vectors with constant heavy and light chain sequences and co-transfected into mammalian Expi293F cells using Expifectamine for micro-expression of IgG. ELISA will be performed to confirm binding of the antibodies to recombinantly expressed antigens. In addition to traditional ELISA, a host protein binding inhibition assay will also be performed to confirm the host protein and the “functionally relevant” antibody isolated from the platform share the same epitope on the antigen.

### Example 3. High-Throughput Simultaneous Identification of Antigen-Antibody Pairs by Screening scFv/Fab Phage Display Library/Antigen-Specific Memory B Cells Using Yeast Antigen Display Library

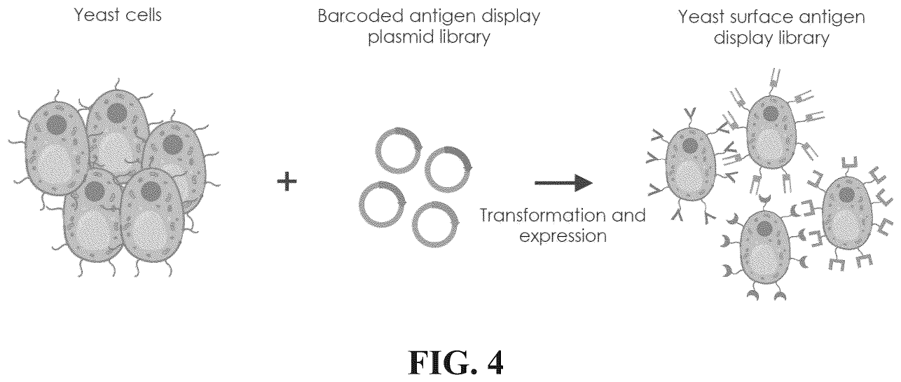

Yeast barcoded antigen display library generation. The first step for the generation of a barcoded antigen display library is the generation of a plasmid library encoding peptide or full-length protein associated with a unique barcode sequencing. FIG. 4 depicts an overview of the generation of a yeast barcoded antigen display libraries. The generation of such a plasmid library can enable the expression of tens of thousands of peptides or proteins in a single reaction including proteins that may be difficult to express recombinantly otherwise. The plasmid library is generated using gateway cloning. Gateway cloning involves the construction of two plasmids—entry clone library and destination vector. The open reading frame (ORF) encoding peptides or full-length proteins are cloned into a pDONR™ vector for the construction of the entry clone library. The entry clone library is generated via a BP reaction involving recombination sites, attB1 and attB2 in the ORFs and recombination sites, attP1 and attP2 in the entry vector using Gateway™ BP clonase™ II enzyme (Invitrogen). The entry clone library has the recombination sites attL1 and attL2. The entry clone library construction can, for example, be used for high-throughput identification of new tumor-associated antigens and neoantigens. In this application, mRNA is extracted from early-stage cancer cells/tissues using Dynabeads™ mRNA DIRECT™ purification kit (Invitrogen). The mRNA is reverse transcribed into cDNA and cloned into pDONR™ vector (example, pDONR222 vector) using the CloneMiner II cDNA library construction kit (Invitrogen). In another application, the entry clone library construction is used for the identification of antigen-antibody pairs associated with nosocomial bacterial infections. In this example, total RNA is extracted from cultures of nosocomial infection causing bacteria such as C. difficile, S. aureus, and A. baumannii using the RNAqueous™ total RNA isolation kit (Invitrogen) and enriched for mRNA from the total RNA using the MICROBExpress™ bacterial mRNA enrichment kit (Invitrogen). Poly(A) tail can then be added to the bacterial mRNA using E. coli Poly(A) polymerase enzyme. The poly(A) tailed mRNA is reverse transcribed into cDNA and cloned into pDONR™ vector (example, pDONR222 vector) using the CloneMiner II cDNA library construction kit (Invitrogen). In yet another example, the entry clone is used for the identification of antigen-antibody pairs associated with HIV-1: synthesized genes corresponding to HIV-1 envelope peptides that have been shown to elicit strong immune response will be synthesized with attB1 and attB2 sites and cloned into the pDONR™ vector.

The ORFs from the entry clone library are cloned into the destination vector via a LR reaction involving recombination sites attL1 and attL2 in the former and recombination sites attR1 and attR2 in the latter using Gateway™ LR clonase™ II enzyme (Invitrogen). Prior to the LR reaction, the destination vector is constructed using a yeast display expression vector (such as the pETcon(−)) as the backbone. Gene fragments encoding the recombination sequence (attR1 and attR2) for insertion of ORF are synthesized and cloned via conventional restriction enzyme digestion and ligation strategy downstream and in frame with the gene coding for Aga2. HA and Myc tags are present upstream and downstream to the attR1 and attR2 sites, respectively. Following gateway cloning of the ORFs, an oligonucleotide pool consisting of a 40 nucleotide long degenerate barcode sequence (for example, (SW)₁₈-AGGA-(SW)₁₈, where S=G/C; W=A/T), complement of 10× template switching oligo (TSO) sequence and complementary of truncated Truseq small RNA read 1 sequence are amplified using polymerase chain reaction (PCR) and assembled into the destination vector, downstream the Gall promoter and upstream the gene coding for Aga2 via Gibson/HiFi DNA assembly. The degeneracy of the oligonucleotide pool can allow for the addition of unique barcodes to the ORFs. The addition of unique barcodes this way can overcome the need for a separate barcode conjugation step of the expressed peptides or proteins. The use of a separate barcode conjugation step usually requires a huge amount of peptides or proteins while having a poor recovery rate. Cloning of the ORFs may also be performed by conventional restriction enzyme digestion and ligation or using Gibson/HiFi DNA assembly into the yeast display expression vectors following the addition of the appropriate flanking sequences. Following cloning of the ORFs, the LR reaction or ligation mix is transformed into E. coli electrocompetent cells (such as TOP10) to obtain the cell surface display plasmid library containing ˜10⁶ independent clones.

scFv/Fab phage display library generation. For scFv (single chain variable fragment) phage display library construction, total RNA is extracted from patient PBMCs (for example, patients with early-stage cancer, patients with past or current infection, or from healthy donors). An overview of the generation of an scFv phage display library from patient PBMCs is shown in FIG. 3. The total RNA is then reverse transcribed into cDNA and subject to PCR to specifically amplify the variable heavy and light chain domains of the B cell receptors (BCRs) and introduce overlapping nucleotide regions for Gibson assembly of the variable heavy and light chains into a phagemid vector (example, pComb3XSS, pAK100) as well as to introduce a linker (example, (G₄S)₃, (G₄S)₄) between the variable heavy and light chains. For vectors that may not have a His tag in their backbone, a His tag is cloned downstream and in-frame to the scFv fragments. The resulting phagemids with scFv are then transformed into electro-competent E. coli cells (for example, TG1 cells) and plated on 2× YT, 2% glucose agar plates with the appropriate antibiotic. The next day, colonies are scraped off using 2× YT media containing glycerol and stored at −80° C. For phage rescue, the glycerol stock is grown to log-phage and infected with 10 times excess of helper phage (example, M13K07). The infected cells are then grown overnight in 2× YT media with appropriate antibiotics for amplification of scFv-displaying phage. The overnight cultures are centrifuged, and the supernatant is collected and mixed with PEG6000/NaCl solution to precipitate phage. The precipitated phage is pelleted and washed with ice-cold DPBS. The washed phage is re-precipitated. The resulting pelleted phage is resuspended in ice-cold DPBS, and centrifuged once more to remove any residual bacteria, and the supernatant is saved as the rescued phage. For Fab (fragment antigen-binding) phage display library constructions, the total RNA is extracted from patient PBMCs or from healthy donors and is reverse transcribed into cDNA. From the cDNA, the heavy chain Fd and k light chain (and/or λ light chain) are specifically amplified and assembled into a phagemid vector. For vectors that may not have a His tag in their backbone, a His tag is cloned downstream and in-frame to the Fab fragments. Transformation into electrocompetent E. coli cells, Fab-expressing phage amplification, and phage rescue are performed as described above.

Antigen-barcode dictionary generation. Following the construction of the plasmid library for yeast display, an antigen-barcode dictionary to identify the barcode associated with each ORF is constructed using Oxford Nanopore long read sequencing approach. The long read sequencing approach can allow for the sequencing of the whole length of the plasmid libraries without the need for assembly. The plasmid library will be prepared for long read sequencing using the Rapid Sequencing kit V14 and loaded onto a R10.4.1 flow cell for sequencing using Nanopore MinION Mk1C sequencer. Basecalling of the generated sequences will be performed using a high accuracy model and subject to bioinformatic analysis using python and R for the construction of the antigen-barcode dictionary. The dictionaries may also be constructed using PacBio or Illumina sequencing when appropriate handles or adapter sequences are added to the oligonucleotide pool consisting of the degenerate barcode sequence.

Antigen-specific FACS. The yeast display plasmid library is transformed into yeast cells (such as EBY100 cells, using Lithium acetate/electroporation/biolistic and glass bead methods) and recovered in YPD media. The yeast cells can then be grown overnight in SDCAA media (30° C., 250 rpm). Yeast cells can also be plated in SDCAA plates to estimate the transformation efficiency. At least 10× the estimated library diversity is pelleted from the overnight culture and resuspended in fresh SDCAA media. The yeast cells are sub-cultured to a concentration of 0.2-0.8×10⁷ cells/ml (OD600=1) in fresh media and grown until the yeast cells reach a mid-log phase (OD600=2-5). The cells can then be subcultured to a concentration of 1×10⁷ cells/ml in SGCAA and incubated at 20° C., 250 rpm for 24-48 hours for surface expression of the antigens. Following the subculture step in SGCAA, the yeast cells can be mixed and incubated with the scFv/Fab phage display library in a rotator at 4° C. Following incubation, the cells are stained with a fluorophore-conjugated antibody (such as FITC-conjugated anti-HA) to the HA tag on the proteins displayed on the yeast cell surface and a fluorophore-conjugated antibody (such as APC anti-His tag antibody) to the His tag on the scFv/Fab expressed on phage. To collect yeast cells bound to phage via antigen-antibody binding, cells that are live and positive for the two fluorophores, one on the anti-myc tag antibody and the other on the anti-His tag antibody are sorted, resuspended in media to 800-1200 cells/μL at a viability of >90%. The sorted cells can then be subject to subsequent library preparation, sequencing, and analysis.

To sort memory B cells expressing antigen-specific antibodies from patient/healthy PBMCs that may bind the antigens on the barcoded yeast display library using FACS, the PBMCs will be stained with a panel of fluorophore-conjugated antibodies specific to cell markers on T cells (CD3), monocytes (CD14) and B cells (CD19, IgG, IgM). The B cells that are live, CD3⁻, CD14⁻, CD19⁺, IgM⁻, IgG⁺ bound to antigen-displaying mammalian cells will be bulk sorted and resuspended in complete RPMI to 800-1200 cells//μL at a viability >90%.

Sequencing and analysis. The sorted cells can then be processed for library preparation using single-cell analysis systems such as 10× Genomics, BD Rhapsody, or others. For library preparation, the sorted cells are partitioned into single-cell suspensions using microfluidic circuits/microwells as per the manufacturer's protocol. The protocol can include a minor modification for the amplification and purification of antigen barcode libraries and scFv/Fab libraries. Briefly, the sorted cells are partitioned into individual cells prior to lysis. Zymolase is added following partition to lyse the yeast cell wall. Upon lysis, the mRNA from the yeast cells is reverse transcribed into cDNA (and also from memory B cells if sorting antigen-specific B cells directly) and the Next Generation Sequencing (NGS) oligo adaptor present on the beads can capture the cDNA and the V_{L}-V_{H} gene from scFv/Fab phagemid/cDNA from memory B cells and add a unique barcode and unique molecular identifiers (UMI) that can allow for the downstream association of the antigen with the scFv/Fab/BCR sequence. Alternatively, polyadenylated sequences on mRNAs and scFv/Fab phagemid (phagemids with cloned polyadenylated sequences) are captured by the beads, after which cDNA synthesis is performed. The cellular cDNA from the yeast cells and the phagemid are amplified using custom primers that flank the cDNA antigen barcodes and the V(D)J genes from BCR/scFv/Fab genes. The resulting amplified libraries are purified using SPRI beads (1.6× purification), and the BCR/scFv/Fab genes and antigen barcode libraries are sequenced using sequencers such as NovaSeq 6000 with a target of at least 10,000 reads per cell. Output fastq files are processed to assemble, quantify, and annotate paired BCR/scFv/Fab sequences and antigen barcodes on a cell-by-cell basis using the barcodes/UMI introduced via the beads. The antigen barcode sequences associated with each single-cell are cross-referenced with the antigen-barcode dictionary to simultaneously discover the antigen that binds a BCR/scFv/Fab. The amplified libraries may also be sequenced using long read sequencing approaches following end repair and appropriate adapter ligation.

Binding score determination. To design the specificity of a BCR/scFv/Fab to its associated antigen, each BCR/scFv/Fab binding an antigen is given a binding score. The binding score is calculated by counting the number of UMIs for the respective scFv/Fab and calculating the centered-log ration (CLR) of each BCR/scFv/Fab UMI for each cell. All counts of 1, 2, and 3 UMI are set to 0 to account for background noise.

Validation and/or characterization of new antigens and antibodies. Antigens and BCR/scFv/Fab to those antigens with a binding score >1 are shortlisted. The variable heavy and light chain sequences of the shortlisted BCR/scFv/Fab are cloned into expression vectors with constant heavy and light chain sequences and co-transfected into mammalian Expi293F cells using a transfection reagent (example, Expifectamine) for micro-expression of IgG. The antigen sequences can also be cloned into expression vectors (for example, pcDNA3.1(+)) and transfected into mammalian Expi293F cells for expression. The recombinantly expressed antigens are purified using affinity chromatography and size exclusion chromatography. The expressed IgGs in supernatants can then be tested for binding to antigens using ELISA. To perform ELISA, the purified antigens are immobilized at a concentration of 2 μg/ml on Nunc immune 96 well plates. Following immobilization, the wells are blocked and incubated with the supernatants containing antibodies. The wells are washed, and the antigen-antibody binding is detected using HRP (horseradish peroxidase)-conjugated anti-human IgG secondary antibody. Data is collected at an absorbance of 450 nm using a plate reader following the addition of the TMB (3,3′,5,5′-Tetramethylbenzidine) substrate.

### Example 4. High-Throughput Simultaneous Identification of Host (Human) Receptor/Protein Associated with Pathogen Invasion/Infection by Yeast Human Receptor/Protein Display Library Using Antigen Yeast Display Library or Recombinantly Expressed Antigens

Yeast barcoded human (host) protein display library generation. The first step for the generation of a barcoded host (human) display library is the generation of a plasmid library encoding peptide or full-length protein associated with a unique barcode sequencing. The generation of such a plasmid library can enable the expression of tens of thousands of peptides or proteins in a single reaction including proteins that may be difficult to express recombinantly otherwise. The plasmid library is generated using gateway cloning. Gateway cloning involves the construction of two plasmids—entry clone library and destination vector. The open reading frame (ORF) encoding peptides or full-length proteins are cloned into a pDONR™ vector for the construction of the entry clone library. The entry clone library is generated via a BP reaction involving recombination sites, attB1 and attB2 in the ORFs and recombination sites, attP1 and attP2 in the entry vector using Gateway™ BP clonase™ II enzyme (Invitrogen). The entry clone library has the recombination sites attL1 and attL2. The entry clone library construction can be used, for example, in the high-throughput identification of host (human) receptors associated with pathogen invasion/infection: cDNA synthesized from total RNA extracted from cultured human mucosal epithelia that form the entry point for most pathogenic organisms, especially viruses is cloned into pDONR™ vector (example, pDONR222 vector) using the CloneMiner II cDNA library construction kit (Invitrogen). The entry clone library can also be the Gateway cloning-adapted CCSB human ORFeome collection. The human ORFeome collection created by the Center for Cancer systems Biology of the Dana-Farber Institute represents more than 12,000 unique genes (including genes coding for cell surface receptors).

The ORFs from the entry clone library are cloned into the destination vector via a LR reaction involving recombination sites attL1 and attL2 in the former and recombination sites attR1 and attR2 in the latter using Gateway™ LR clonase™ II enzyme (Invitrogen). Prior to the LR reaction, the destination vector is constructed using a yeast display expression vector (such as the pETcon(−)) as the backbone. Gene fragments encoding the recombination sequence (attR1 and attR2) for insertion of ORF are synthesized and cloned via conventional restriction enzyme digestion and ligation strategy downstream and in frame with the gene coding for Aga2. HA and Myc tags are present upstream and downstream to the attR1 and attR2 sites, respectively. Following gateway cloning of the ORFs, an oligonucleotide pool consisting of a 40 nucleotide long degenerate barcode sequence (for example, (SW)₁₈-AGGA-(SW)₁₈, where S=G/C; W=A/T), complement of 10× template switching oligo (TSO) sequence and complementary of truncated Truseq small RNA read 1 sequence are amplified using polymerase chain reaction (PCR) and assembled into the destination vector, downstream the Gal1 promoter and upstream the gene coding for Aga2 via Gibson/HiFi DNA assembly. The degeneracy of the oligonucleotide pool can allow for the addition of unique barcodes to the ORFs. The addition of unique barcodes this way can overcome the need for a separate barcode conjugation step of the expressed peptides or proteins. The use of a separate barcode conjugation step usually requires a huge amount of peptides or proteins while having a poor recovery rate. Cloning of the ORFs may also be performed by conventional restriction enzyme digestion and ligation or using Gibson/HiFi DNA assembly into the yeast display expression vectors following the addition of the appropriate flanking sequences. Following cloning of the ORFs, the LR reaction or ligation mix is transformed into E. coli electrocompetent cells (such as TOP10) to obtain the cell surface display plasmid library containing ˜10⁶ independent clones.

Host protein-Barcode dictionary generation. Following the construction of the plasmid library for yeast display of human (host) proteins, a host protein-barcode dictionary to identify the barcode associated with each ORF is constructed using Oxford Nanopore long read sequencing approach. The long read sequencing approach can allow for the sequencing of the whole length of the plasmid libraries without the need for assembly. The plasmid library will be prepared for long read sequencing using the Rapid Sequencing kit V14 and loaded onto a R10.4.1 flow cell for sequencing using Nanopore MinION Mk1C sequencer. Basecalling of the generated sequences will be performed using a high accuracy model and subject to bioinformatic analysis using python and R for the construction of the host protein-barcode dictionary. The dictionaries may also be constructed using PacBio or Illumina sequencing when appropriate handles or adapter sequences are added to the oligonucleotide pool consisting of the degenerate barcode sequence.

Barcoded Antigen yeast display library generation. For the generation of a barcoded antigen yeast surface display library, an oligonucleotide pool with degenerate barcode sequence (e.g., WSWSWSWSWSWSWSWSWSAGGAWSWSWSWSWSWSWSWSWS, where W=A/T, S=G/C), complement of 10× template switching oligo (TSO) sequence and complement of truncated Truseq small RNA read 1 sequence will be amplified using polymerase chain reaction (PCR) and assembled into the yeast surface display vector such as pETcon(−) using Gibson assembly. Prior to insertion of the barcode, attR1 and attR2 sites for gateway cloning of antigen genes (for example, viral proteins) will be inserted into the vector. Following gateway cloning of the antigen genes, the barcodes is cloned into the plasmid library via Gibson/HiFi DNA assembly. An antigen-barcode dictionary like the host protein-barcode dictionary will be generated. The barcoded yeast surface antigen display plasmid library are then transformed into yeast cells such as S. cerevisiae EBY100 and induced for surface expression of the antigens in galactose-rich selective media. The antigens will be expressed as C-terminal fusion proteins to the anchor yeast protein Aga2p. An epitope tag such as a His tag on the fusion proteins will allow detection of surface expression in FACS using a fluorophore conjugated anti-His antibody.

Barcoded antigen library can also be prepared by recombinant expression. Plasmids encoding Avi-tagged antigens is transiently transfected into Expi293F cells using Gibco™ ExpiFectamine™ 293 transfection kit for expression. The constructs will have a His epitope tag for detection downstream. The expressed antigens are then purified from the cell culture supernatants using affinity chromatography and size exclusion chromatography. The purified Avi-tagged antigens are biotinylated using BirA enzyme. Streptavidin-PE linked to a unique oligonucleotide/DNA barcode are conjugated to the antigens via biotin.

Antigen-specific FACS. The yeast display plasmid libraries are transformed into yeast cells (such as EBY100 cells, using Lithium acetate/electroporation/biolistic and glass bead methods) and recovered in YPD media. The yeast cells can then be grown overnight in SDCAA media (30° C., 250 rpm). Yeast cells can also be plated in SDCAA plates to estimate the transformation efficiency. At least 10× the estimated library diversity is pelleted from the overnight culture and resuspended in fresh SDCAA media. The yeast cells are sub-cultured to a concentration of 0.2-0.8×10⁷ cells/ml (OD600=1) in fresh media and grown until the yeast cells reach a mid-log phase (OD600=2-5). The cells are then sub-cultured to a concentration of 1×10⁷ cells/ml in SGCAA and incubated at 20° C., 250 rpm for 24-48 hours for surface expression of the host proteins/antigens. The antigen-expressing yeast cells/barcoded recombinantly expressed antigens are mixed and incubated with the yeast host protein display library in a rotator at 4° C. Following incubation, the cells are stained with a fluorophore-conjugated antibody (such as FITC-conjugated anti-HA antibody) to the HA tag on the host proteins on the mammalian cell surface and a fluorophore-conjugated antibody (such as APC anti-His tag antibody) to the His tag on the yeast cells expressing antigens (in the case of recombinantly expressed antigens, they are already conjugated to PE). To collect host protein expressing yeast cells bound to antigen expressing yeast cells/recombinant antigen via host protein-antigen binding, cells that are live and positive for the two fluorophores, one on the anti-HA tag antibody and the other on the anti-His tag antibody (or recombinant antigen) are sorted, resuspended in YPD media to 800-1200 cells/μL at a viability of >90%. The sorted cells will then be subject to subsequent library preparation, sequencing, and analysis.

For high-throughput simultaneous identification of host (human) receptors and potently neutralizing antibodies against the pathogen via ligand blocking, fluorophore-stained PBMCs from patients with active infection and/or healthy individuals previously exposed to the pathogen is mixed with the barcoded antigen library and the barcoded host protein library. Memory B cells (CD3⁻, CD14⁻, CD19⁺, IgM⁻, IgG⁺) bound to the antigens as well as to host (human) protein-antigen complexes are sorted. The rationale for sorting the two populations is that when an antibody binds to the antigen alone but not to the complex, the antibody might be binding to an epitope that prevents the interaction between the antigen and the host protein.

Sequencing and analysis. The sorted cells can then be processed for library preparation using single-cell analysis systems such as 10× Genomics, BD Rhapsody, or others. For library preparation, the sorted cells are partitioned into single-cell suspensions using microfluidic circuits/microwells as per the manufacturer's protocol. The protocol will include a minor modification for the amplification and purification of human protein barcode libraries and antigen yeast libraries/recombinantly expressed and barcoded antigens. Briefly, the sorted cells are partitioned into individual cells prior to lysis. Zymolase is added for lysis of yeast cells. Upon lysis, the mRNA from the yeast cells is reverse transcribed into cDNA and the Next Generation Sequencing (NGS) oligo adaptor present on the beads can capture the cDNA (and oligonucleotide barcode on the recombinantly expressed antigens) and add a unique barcode and unique molecular identifiers (UMI) that can allow for the downstream association of human protein with antigen sequence. Alternatively, polyadenylated sequences on mRNAs are captured by the beads, after which cDNA synthesis is performed. The cellular cDNA from the yeast cells/oligonucleotide barcodes on recombinant antigens are amplified using custom primers that flank the cDNA host protein barcodes and antigen barcodes/oligonucleotide barcodes. The resulting amplified libraries are purified using SPRI beads (1.6× purification), and the antigens barcodes/oligonucleotide barcodes and human protein barcode libraries are sequenced using sequencers such as NovaSeq 6000 with a target of at least 10000 reads per cell. Output fastq files are processed to assemble, quantify, and annotate paired antigen sequences and human protein barcodes on a cell-by-cell basis using the barcodes/UMI introduced from the beads. The human protein barcode sequences associated with each single-cell are cross-referenced with the human protein-barcode dictionary to simultaneously discover the human protein the antigens can bind. The amplified libraries may also be sequenced using long read sequencing approaches following end repair and appropriate adapter ligation. For high-throughput simultaneous identification of host (human) receptors and potently neutralizing antibodies against the pathogen via ligand blocking, the cellular cDNA from B cells will be amplified using primers that flank the V(D)J genes in the B cell receptor (BCR). The oligonucleotide with the antigen barcode and cDNA with the host protein barcode will be amplified using custom primers. Following purification and sequencing of the antibodies, antigen and host protein barcodes, the output fastq files will be processed using Cell Ranger to assemble, quantify and annotate the barcode sequences on a cell-by-cell basis.

Binding score determination. To design the specificity of an antigen to its associated human protein/receptor, each antigen binding to a human protein is given a binding score. The binding score is calculated by counting the number of UMIs for the respective antigen and calculating the centered-log ratio (CLR) of each antigen UMI for each cell. All counts of 1, 2, and 3 UMI are set to 0 to account for background noise. With regards to high-throughput simultaneous identification of host (human) receptors and potently neutralizing antibodies against the pathogen via ligand blocking, for each antibody, antigen barcode UMI count matrix and host protein barcode UMI count matrix is be created and CLR is calculated. This allows the identification of antigen-host receptor pairs, along with both antibodies that are targeting epitopes outside of the respective receptor/protein binding site, as well as antibodies that can block the antigens from interacting with the respective host proteins.

Validation and/or characterization of antigens that may bind human proteins. Antigens and human proteins binding with a score >1 are shortlisted. The antigen sequences (as gBlock gene fragments, for instance) are cloned into expression vectors (example, pcDNA3.1(+)) and transfected into mammalian Expi293F cells using a transfection reagent (example, Expifectamine) for recombinant expression of antigens. In a similar way, host (human) proteins can also be recombinantly expressed. The recombinantly expressed antigens and human proteins are purified using affinity chromatography and size exclusion chromatography. The expressed antigens can then be tested for binding to human proteins using ELISA. To perform ELISA, the purified antigens are immobilized at a concentration of 2 μg/ml on Nunc immune 96 well plates. Following immobilization, the wells are blocked and incubated with the human proteins. The wells are washed, and the antigen-human protein binding is detected using an anti-tag antibody (such as anti-His, based on the tag on the human protein) conjugated to HRP. Data is collected at an absorbance of 450 nm using a plate reader following the addition of the TMB substrate. With regards to high-throughput simultaneous identification of host (human) receptors and potently neutralizing antibodies against the pathogen via ligand blocking, the variable heavy and light chain sequences of the shortlisted antibodies is cloned into expression vectors with constant heavy and light chain sequences and co-transfected into mammalian Expi293F cells using Expifectamine for micro-expression of IgG. ELISA will be performed to confirm binding of the antibodies to recombinantly expressed antigens. In addition to traditional ELISA, a host protein binding inhibition assay will also be performed to confirm the host protein and the “functionally relevant” antibody isolated from the platform share the same epitope on the antigen.

### Example 5. High Throughput Identification of Antibodies to Peptides from HIV-1 Envelope

From the HIV molecular immunology database, 48 HIV-1 envelope linear epitope sequences were chosen for expression as HIV-1 env peptides on mammalian/yeast surface. The linear epitopes were chosen based on their immunogenicity and availability of known antibodies to the region. Gene fragments to be synthesized as gBlocks (IDT) were designed with flanking attB1 and attB2 sites and a nucleotide sequence of the (G₄S)₃ linker upstream the peptide sequence. The attB1 and attB2 sites were added for gateway cloning of the genes coding for the peptides into the entry clone vector pDONR221 for subsequent cloning into the mammalian/yeast surface display vector (pCMVFRTDEST/pETcon.gateway, respectively). An overnight BP clonase reaction was performed for cloning of the gene fragments into the pDONR221 vector. Following clonase reaction, the reaction mix was transformed into One Shot Top10 chemically competent cells and spread on LB agar plates supplemented with Kanamycin. Following overnight incubation, single colonies were picked, and plasmid was extracted using plasmid mini prep kits. The purified plasmids were then pooled and cloned into the mammalian/yeast surface display vector using an overnight LR clonase reaction. Following LR clonase reaction, the reaction mix was subject to clean-up using PCR and DNA clean-up kit and the entire mix was transformed into Electromax DH10B competent cells. The transformed bacterial cells were plated onto LB agar plates supplemented with ampicillin and following overnight incubation, all bacterial colonies were scrapped from the plates and subject to plasmid extraction. The resulting HIV peptides mammalian display plasmid library was linearized, and PCR amplified barcode library was inserted via HiFi DNA assembly. The assembly reaction was cleaned-up with PCR and DNA clean-up kit and the entire mix was transformed into NEB 10-beta electrocompetent cells. The transformed bacterial cells were plated onto LB agar plates supplemented with ampicillin and following overnight incubation, all bacterial colonies except for four single colonies were scrapped from the plates and subject to plasmid extraction. The four single colonies were separately inoculated into LB media supplemented with ampicillin for plasmid extraction. Sequencing of the extracted plasmids confirmed cloning of unique barcodes into the HIV peptides mammalian display plasmid library (pCMVFRTDEST.HIVpep)—sequence verification results are shown as SEQ ID NOs. 1-4. The nucleotides sequences of the four HIV peptides and their respective barcodes are shown in SEQ ID NOs. 5-8 and 9-12, respectively. Colony count on a plate spread with 200 μl transformed bacteria was approximately 350. Hence, the library size has been calculated to be approximately 175,000 when 1 μg of assembled plasmid library is transformed. The barcoded HIV peptides mammalian display plasmid library will next be subject to Oxford Nanopore long read sequencing and analysis for the construction of the HIVpeptide-barcode dictionary. Test co-transfection of a single barcoded HIV peptide mammalian display plasmid into Flp-In 293 along with pOG44 plasmid for stable integration of the HIV peptide may be done to confirm surface expression of the peptide using flow cytometry. Once the proposed surface display system is proven to be successful, the barcoded plasmid library will be transfected into the Flp-In 293 cells for surface expression of the 48 HIV-1 env peptides and mixed with PBMCs from HIV patients/scFv/Fab phage display library generated from PBMCs of HIV patients for sorting of antigen-antibody pairs. The sorted cells are then processed for sequencing to identify the antigen-antibody pairs. For yeast display of HIV peptides, the entry clone vector with the 48 HIV peptides have been gateway cloned into the custom pETcon.gateway vector.

### Example 6. High-Throughput Epitope Mapping of Broadly Neutralizing Antibodies that Bind BG505

Some known broadly neutralizing antibodies (bnAbs) against the HIV env, BG505 binding the V3 loop such as PGT121 and PGT128 have been well-characterized. The present examples aims to uniquely barcode the two bnAbs and screen against a high-throughput barcoded BG505 alanine scanning mutagenesis library expressed on the surface of mammalian cells using flow cytometry and utilize next-generation sequencing to identify the mutant BG505 env proteins the antibodies do and do not bind. This information will aid in the determination of the epitopes. The ability to precisely identify the epitopes of the already well-characterized bnAbs indicates the ability of the technology to map antibodies binding BG505.

The first step in the generation of the high-throughput barcoded BG505 mutant display library involves the construction of a mammalian surface display vector followed by cloning of an alanine scanning mutagenesis library of the BG505.SOSIP v9.3 construct. An alanine scanning mutagenesis library of the BG505.SOSIP v9.3 construct was generated by first cloning the construct into the pDONR221 vector and then introducing point alanine mutations in each residue of the V3 loop (alanine on the loop was changed to serine). Following mutagenesis, the BG505.SOSIP v9.3 constructs with the V3 loop alanine scanning mutations were cloned into the mammalian surface display vector (pCMVFRTDEST) by gateway cloning. Alanine scanning library generation was performed by GenScript. To confirm alanine substitution of the residues in the V3 loop, the library was transformed into One shot Top10 chemically competent cells and spread on LB agar plates supplemented with ampicillin. Single colonies were picked and inoculated into LB media supplemented with ampicillin for bacterial propagation and plasmid extraction. The extracted plasmids were then sequence verified for the presence of all variants. The sequences of the 33 BG505.SOSIP v9.3 V3 loop variants in the mammalian display vector are shown in SEQ ID NOs. 13-45. The mammalian display BG505.SOSIP v9.3 V3 loop alanine scanning plasmid library was linearized, and PCR amplified barcode library was inserted via HiFi DNA assembly. The assembly reaction was cleaned-up with PCR and DNA clean-up kit and the entire mix was transformed into NEB 10-beta electrocompetent cells. The transformed bacterial cells were plated onto LB agar plates supplemented with ampicillin and following overnight incubation, all bacterial colonies except for six single colonies were scrapped from the plates and subject to plasmid extraction. The six single colonies were separately inoculated into LB media supplemented with ampicillin for plasmid extraction. Sequencing of the extracted plasmids confirmed cloning of unique barcodes into the mammalian display BG505.SOSIP v9.3 V3 loop alanine scanning plasmid library-sequence verification results are shown in SEQ ID NOs. 46-51. The six BG505.SOSIP v9.3 V3 alanine mutations and their respective barcodes are shown in SEQ ID NOs. 52-69. Colony count on a plate spread with 200 μl transformed bacteria was approximately 251. Hence, the library size has been calculated to be approximately 125,500 when 1 μg of assembled plasmid library is transformed. The barcoded BG505.SOSIP v9.3 V3 loop alanine scanning mammalian display plasmid library will next be subject to Oxford Nanopore long read sequencing and analysis for the construction of the BG505.SOSIP v9.3 V3 loop alanine mutation-barcode dictionary. Following the construction of the dictionary, the barcoded plasmid library will be stably co-transfected into Flp-In 293 cells alone with pOG44 for integration of the ORF into the genome of the cells. The two antibodies PGT121 and PGT128 have already been obtained from BEI resources. The two antibodies are then coupled to unique oligonucleotide barcodes and fluorophores by site-specific conjugation for sorting of the BG505.SOSIP v9.3 V3 loop alanine mutation expressing mammalian cells. Mammalian cells expressing the mutant BG505 env proteins will have a HA antibody and are incubated with the fluorophore-conjugated anti-HA antibody. The mammalian cells are then incubated with each fluorophore-conjugated bnAb. Cells that are positive for the fluorophore on the anti-HA antibody and the fluorophore the bnAbs will be sorted. Following downstream processing, sequencing and analysis, the mutant BG505 sequences that bnAbs bind thereto can be determined. This way the specific residues (epitope) for the binding of the bnAbs can be mapped. The mapped epitopes are then compared with the already known epitopes of the two bnAbs to confirm accuracy of this approach.

### Example 7. High-Throughput Mapping of Viral Antigens to Their Host Receptors

The present example describes host receptor discovery for a set of diverse coronaviruses that are both known and unknown to infect humans. Even though different coronaviruses have been implicated in interaction with several different host receptors, the receptors for only a small subset of coronaviruses are currently known. This family of viruses is therefore an excellent target for the disclosed technology since it both provides confirmed positive controls (for the known virus-receptor pairs) and an opportunity for new virus-receptor discovery. The successful application of the technology in this example can be expanded to produce a general platform for simultaneous virus antigen-host receptor discovery. These efforts will offer an unprecedented opportunity to understand the fundamentals of virus-host interactions at an unparalleled scale and across host species and will also enable the development of novel therapeutic and preventive modalities, as a first line of defense against both current and emerging viruses.

The first step in the generation of the high-throughput barcoded human host protein display library is the cloning of the gateway-adapted CCSB human ORFeome collection into the custom mammalian display vector (pCMVFRTDEST). Given the ORFeome collection is expensive, the study sought to amplify certain genes from cultured healthy and diseases epithelial cells in-house and add attB1 and attB2 sites for cloning into pDONR221 vector. Total RNA from primary healthy bronchial epithelial cells was isolated using RNeasy mini prep kit. cDNA was synthesized from the total RNA using superscript IV reverse transcriptase enzyme with oligodT primer. The synthesized cDNA was then used as the template for PCR amplification of 13 different genes known to be expressed in well expressed in healthy epithelial cells. These 13 genes include genes coding for host proteins for non-coronaviruses viruses as well as genes for amplification control. For amplification of the genes, primers were designed based on the annotated sequences of the human genome (hORFeome v9.1). The primer sequences for amplification of the genes as well as addition of attB1 and attB2 sites are shown in SEQ ID NOs. 70-99. Following PCR amplification of the 13 genes, the amplification reaction was run in a DNA gel and gel purified. The gel purified amplified genes were gateway cloned into pDONR221 and sequence verified. The sequence verification results are shown in SEQ ID NOs. 100-112. Calu-3, a well-established cell line from the airway epithelia of a patient with lung adenocarcinoma are currently in culture for the isolation of the two genes encoding host proteins known to be associated with entry of coronaviruses such as ACE2 and DPP4. Following amplification and cloning of ACE2 and DPP4, all 15 genes in the entry vector will be cloned into the custom mammalian display vector (pCMVFRTDEST) and barcoded. The resulting barcoded plasmid library will be transfected into Flp-In 293 cells for surface expression of the 15 host proteins. The mammalian cells will then be mixed with recombinantly expressed and barcoded antigens such as SARS-COV-2, MERS and SARS-COV-1 spike proteins and sorted. The sorted cells will be processed, sequenced and analyzed to find host protein-antigen pairs. The ability of the platform to identify known binding pairs: SARS-COV-2 spike-ACE2, SARS-COV-1 spike-ACE2 and MERS-DPP4 confirms the platforms ability to map coronavirus antigens to their host receptor. The platform can also be expanded to include yeast antigen display library for screening of the host protein-antigen pairs.

As an alternate approach, construction of gateway-adapted cDNA library from mRNA extracted from cultured epithelial cells using Cloneminer II cDNA library construction kit may also be used.

### Example 8. High-Throughput Identification of Antigen-Antibody Pairs Associated with S. aureus Infection

Currently there are no vaccines to prevent nosocomial infections caused by bacteria such as antibiotic-resistant Staphylococcus aureus, Acinetobacter baumannii and Clostridioides difficile. A successful vaccine to protect against these infections will need to target multiple antigens on the bacteria. Using the disclosed technology, the study sought to capture antibody-antigen pairs from PBMCs isolated from patients with active infection and/or patients who have recovered from these infections. The sequence information of the captured antibody-antigen pairs from the approach will enable the identification of previously unidentified bacterial proteins involved in eliciting a strong immune response in humans that may then be utilized for the design of better vaccine candidates. The isolated antibodies can be utilized for prophylaxis or as a therapeutic against these bacterial infections.

Towards this aim, total RNA have been isolated from lab grown cultures of S. aureus using the RNAqueous total RNA isolation kit and enriched for mRNA from the total RNA using the MICROBExpress bacterial mRNA enrichment kit. Poly(A) tail was then added to the bacterial mRNA using E. coli poly(A) polymerase enzyme. The poly(A) tailed mRNA was reverse transcribed into mRNA and cloned into pDONR222 vector using the CloneMiner II cDNA library construction kit for subsequent gateway cloning into the custom mammalian/yeast surface display vector (pCMVFRTDEST/pETcon.gateway vector). Following cloning into the pDONR222 vector, the BP clonase reaction mix was transformed into Electromax DH10B competent cells and spread on LB agar plates supplemented with Kanamycin. Following overnight incubation, all bacterial colonies were scrapped from the plates and subject to plasmid extraction. The extracted plasmids were then pooled and cloned into the mammalian/yeast surface display vector using an overnight LR clonase reaction. Following LR clonase reaction, the reaction mix was subject to clean-up using PCR and DNA clean-up kit and the entire mix was transformed into Electromax DH10B competent cells. The transformed bacterial cells were plated onto LB agar plates supplemented with ampicillin and following overnight incubation, all bacterial colonies were scrapped from the plates and subject to plasmid extraction. The plasmids were pooled, and the the resulting plasmid library is ready for barcoding.

A combinational scFv phage display library construction can also be formed. From PBMCs collected from patients post 2-4 weeks of S. aureus infection, total RNA was extracted, and cDNA was synthesized using superscript IV reverse transcriptase and oligo dT primer. The variable heavy and light chain sequences (V_{H} and V_{L}) were specifically amplified using a degenerate set of primers that were also modified to include overlapping sequencing between the vector and the genes and between the genes to assemble the variable genes into the linearized vector using Gibson assembly. The primers are shown in SEQ ID NOs. 113-156. The vector was modified at the overlapping nucleotide region from the original pComb3XSS sequence to reduce palindromic sequences and increase the efficiency of Gibson assembly of the variable genes. The modified pComb3XSS vector was linearized using SacI and SpeI restriction enzymes and assembly reaction was set up with the linearized vector and the amplified VH and VL genes. Following assembly, the reaction was transformed into TG1 electrocompetent cells and spread on LB agar plate supplemented with ampicillin. About 10 single colonies from the plate following overnight incubation were picked and cultured. Plasmids were extracted from the cultures were sent for sequence verification to confirm insertion of the genes in the form of scFv into the modified pComb3XSS vector. From the sequencing results, 6 out of 10 had the scFv insertions. Hence, the efficiency of scFv assembly was around 60%. The sequence verification results are shown in SEQ ID NOs. 157-166. More plasmids can also be transformed into TG1 cells for subsequence phage infection and phage display library generation.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method for simultaneous detection of an antigen and an antibody that specifically binds said antigen, comprising:
contacting a population of eukaryotic cells engineered to display a plurality of barcode-labeled antigens with a phage display library expressing a plurality of fragment antigen-binding (Fab) regions of an antibody;
allowing the plurality of barcode-labeled antigens to bind to the plurality of Fab regions of the phage to form an antibody-antigen binding complex comprising eukaryotic cells bound to phages;
separating the antibody-antigen binding complex into single-cell partitions;
introducing into each single-cell partitions a unique cell barcode-labeled bead;
preparing a single-cell cDNA library from the single-cell partitions;
performing PCR amplification reactions to produce a plurality of amplicons, wherein the amplicons comprise: 1) the cell barcode and the antigen barcode, 2) the cell barcode and a paired Fab sequence, and 3) a unique molecular identifier (UMI); and
sequencing the plurality of amplicons.

2. The method of claim 1, wherein the eukaryotic cells are mammalian cells.

3. The method of claim 2, wherein the mammalian cells are HEK293T cells and/or CHO cells.

4. The method of claim 1, wherein the eukaryotic cells are yeast cells.

5. The method of claim 4, wherein the yeast cells are Saccharomyces cerevisiae cells.

6. The method of claim 1, wherein the barcode-labeled antigens are labeled with a first barcode comprising a DNA sequence or an RNA sequence and the cell barcode-labeled beads are labeled with a second barcode comprising a DNA sequence or an RNA sequence.

7. The method of claim 1, wherein the barcode-labeled antigens comprise an antigen from a pathogen or an animal.

8. The method of claim 7, wherein the antigen from the animal comprises a tumor-associated antigen or a neoantigen.

9. The method of claim 8, wherein the antigen from a pathogen comprises an antigen from a nosocomial infection causing bacteria.

10. The method of claim 9, wherein the nosocomial infection causing bacteria comprises C. difficile, S. aureus, A. baumannii, or a combination thereof.

11. The method of claim 1, wherein the plurality of antigens comprise a panel of epitope knock-outs.

12. The method of claim 1, wherein the plurality of antigens comprises a panel of antigen variants or mutations for epitope mapping.

13. The method of claim 1, wherein the phage display expresses a plurality of natively or combinational paired single chain variable fragments (scFvs).

14. The method of claim 1, further comprising determining a binding score by counting the number of UMIs for each paired Fab sequence.

15. A method for simultaneous detection of an antigen and an antibody that specifically binds said antigen, comprising:
contacting a population of eukaryotic cells engineered to express a plurality of barcode-labeled proteins with a yeast display library expressing a plurality of antigens;
allowing the plurality of barcode-labeled proteins to bind to the plurality of antigens of the yeast display library to form a protein-antigen binding complex comprising eukaryotic cells bound to yeast cells;
separating the protein-antigen binding complex into single-cell partitions;
introducing into each single-cell partitions a unique cell barcode-labeled bead;
preparing a single-cell cDNA library from the single-cell partitions;
performing PCR amplification reactions to produce a plurality of amplicons, wherein the amplicons comprise: 1) the cell barcode and the protein barcode, 2) the cell barcode and an associated protein and antigen sequence, and 3) a unique molecular identifier (UMI); and
sequencing the plurality of amplicons.

16. The method of claim 15, wherein the barcode-labeled proteins are labeled with a first barcode comprising a DNA sequence or an RNA sequence, and the cell barcode-labeled beads are labeled with a second barcode comprising a DNA sequence or an RNA sequence.

17. The method of claim 15, wherein the barcode-labeled proteins comprise a receptor proteins associated with viral infection.

18. The method of claim 17, wherein receptor proteins comprise human receptor proteins.

19. The method of claim 18, wherein the human receptor proteins comprise proteins from human epithelial cells.

20. A method for simultaneous detection of an antigen and an antibody or binding fragment thereof that specifically binds said antigen, the method comprising:
contacting a population of eukaryotic cells engineered to display a plurality of barcode-labeled antigens with a population of antigen-specific B cells;
allowing the plurality of barcode-labeled antigens to bind to the population of antigen-specific B cells to form an antibody-antigen binding complex comprising eukaryotic cells bound to B cells;
separating the antibody-antigen binding complex into single-cell partitions (e.g., droplet-based or well-based assays);
introducing into each single-cell partitions a unique cell barcode-labeled bead;
preparing a single-cell cDNA library from the single-cell partitions;
performing PCR amplification reactions to produce a plurality of amplicons, wherein the amplicons comprise: 1) the cell barcode and the antigen barcode, 2) the cell barcode and a paired antibody or antibody binding fragment sequence, and 3) a unique molecular identifier (UMI); and
sequencing the plurality of amplicons.
